SEQUENCE LISTING (28 sequences)

SEQ ID NO: 1 - DNA, 28 bp, Artificial Sequence
  Description of Artificial Sequence Synthetic primer
ggaattcatgcaagatgaaaaaaacgga

SEQ ID NO: 2 - DNA, 48 bp, Artificial Sequence
  Description of Artificial Sequence Synthetic primer
ggaagctttcagtgatggtgatggtgatgttccggctcccaacaattt

SEQ ID NO: 3 - DNA, 36 bp, Artificial Sequence
  Description of Artificial Sequence Synthetic primer
aagaacttaaccataagcggaccagtggaggcactc

SEQ ID NO: 4 - DNA, 36 bp, Artificial Sequence
  Description of Artificial Sequence Synthetic primer
gagtgcctccactggtccgcttatggttaagttctt

SEQ ID NO: 5 - DNA, 32 bp, Artificial Sequence
  Description of Artificial Sequence Synthetic primer
gtggaattctaatcgaggcccttgtgaaggac

SEQ ID NO: 6 - DNA, 29 bp, Artificial Sequence
  Description of Artificial Sequence Synthetic primer
cacgtcgacttcagccggccactgctggg

SEQ ID NO: 7 - DNA, 32 bp, Artificial Sequence
  Description of Artificial Sequence Synthetic primer
ccggatccaccatggaaactaaacctcccaca

SEQ ID NO: 8 - DNA, 31 bp, Artificial Sequence
  Description of Artificial Sequence Synthetic primer
ccgcggccgcttaataagactgaacgaggct

SEQ ID NO: 9 - DNA, 29 bp, Artificial Sequence
  Description of Artificial Sequence Synthetic primer
ccaagcttatgcaagatgaaaaaaacgga

SEQ ID NO: 10 - DNA, 29 bp, Artificial Sequence
  Description of Artificial Sequence Synthetic primer
ctgaagctttggtgtgggtgtgtgggtag

SEQ ID NO: 11 - DNA, 24 bp, Artificial Sequence
  Description of Artificial Sequence Synthetic primer
tccggatccaacaggaggaagtat

SEQ ID NO: 12 - DNA, 24 bp, Artificial Sequence
  Description of Artificial Sequence Synthetic primer
cacgaattcctgcaggttgtccga

SEQ ID NO: 13 - DNA, 23 bp, Artificial Sequence
  Description of Artificial Sequence Synthetic primer
gccggtcctccctggagaactcc

SEQ ID NO: 14 - DNA, 27 bp, Artificial Sequence
  Description of Artificial Sequence Synthetic primer
tccggactcagatctatggcccagggg

SEQ ID NO: 15 - DNA, 27 bp, Artificial Sequence
  Description of Artificial Sequence Synthetic primer
gtctgtgttaagcttttccggctccca

SEQ ID NO: 16 - DNA, 27 bp, Artificial Sequence
  Description of Artificial Sequence Synthetic primer
tccggactcagatctatggcccagggg

SEQ ID NO: 17 - DNA, 21 bp, Artificial Sequence
  Description of Artificial Sequence Synthetic primer
cccaagcttttctttgcttgc

SEQ ID NO: 18 - protein, 6 aa, Artificial Sequence
  Description of Artificial Sequence Synthetic 6x His tag
HHHHHH

SEQ ID NO: 19 - protein, 87 aa, Saccharomyces cerevisiae
LSQVAVEFRKRLNIGDRVKDGLLYKDAFLGSEAVDVLMHIVRTTDRNLALLLGRALDSQKMFHDVTYSHRLRDSLKEVYQYRRIISP

SEQ ID NO: 20 - protein, 86 aa, Saccharomyces cerevisiae
LQLLLTKLPKTDYKLPLISYSLSNTNNGGEITKFLLDHMSLKDIDQAETFGQDLLNLGFLKYCNGVGNTFVNSKKFQYQWKNTAYM

SEQ ID NO: 21 - protein, 89 aa, Homo sapiens
MASVTKAMAAPESGLEVRDRMWLKITIPNAFLGSDVVDWLYHHVEGFPERREARKYASGLLKAGLIRHTVNKITFSEQCYYVFGDLSGG

SEQ ID NO: 22 - protein, 89 aa, Homo sapiens
MSAVVRVMQLPDSGLEIRDRMWLKITIANAVIGADVVDWLYTHVEGFKERREARKYASSLLKHGFLRHTVNKITFSEQCYYVFGDLCSN

SEQ ID NO: 23 - protein, 90 aa, Bos sp.
MEDVIARMQDEKNGIPIRTVKSFLSKIPSVFSGSDIVQWLIKNLTIEDPVEALHLGTLMAAHGYFFPISDHVLTLKDDGTFYRFQTPYFW

SEQ ID NO: 24 - protein, 91 aa, Homo sapiens
IEDIITKMQDDKTGGVPIRTVKSFLSKIPSVVTGTDIVQWLMKNLSIEDPVEAIHLGSLIAAQGYIFPISDHVLTMKDDGTFYRFQAPYFW

SEQ ID NO: 25 - protein, 90 aa, Caenorhabditis elegans
MEMLVNQMLDAEAGVPIKTVKSFLSKVPSVFTGQDLIGWIMKNLEMTDLSDALHLAHLIASHGYLFQIDDHVLTVKNDGTFYRFQTPYFW

SEQ ID NO: 26 - protein, 90 aa, Bos sp.
IEALVKDMQDPDTGVRVQNQKVKVVSIPHAMTGSDVLQWISQRLWISGLEAQNLGNFIVKYGYIYPLQDPRNLTLKPDSSLYRFQTPYFW

SEQ ID NO: 27 - protein, 90 aa, Homo sapiens
MERVVVSMQDPDQGVKMRSQRLLVTVIPHAVTGSDVVQWLAQKFCVSEEEALHLGAVLVQHGYIYPLRDPRSLMLRPDETPYRFQTPYFW

SEQ ID NO: 28 - protein, 87 aa, Caenorhabditis elegans
IEVLAKRVMEGAQLKTHKYFRIAVPHAITGQQLIALVLERGAPDDEAEASHLASLLLHHGYMFPVIEHGQPFKDDGTLYRLQRPYFW

(19) United States
(12) Patent Application Publication
(10) Pub. No.: US 2010/0183584 A1
(43) Pub. Date: Jul. 22, 2010

(54) CONFORMATION AND ACTIVITY OF GBETA5 COMPLEXES

(75) Inventors: Vladlen Slepak, Pinecrest, FL (US); Simone Sandiford, Miami, FL (US); Qiang Wang, Miami, FL (US)

Correspondence Address: NOVAK DRUCE + QUIGG LLP (WPB), 525 Okeechobee Blvd, 15th Floor, City Place Tower, West Palm Beach, FL, 33401, US

(73) Assignee: University Of Miami, Miami, FL (US)

(21) Appl. No.: 12/508,376

(22) Filed: Jul. 23, 2009

Related U.S. Application Data
(63) Continuation-in-part of application No. PCT/US2008/000819, filed on Jan. 23, 2008.
(60) Provisional application No. 60/881,847, filed on Jan. 23, 2007.

(51) Int. Cl. A61K 39/395; C12Q 1/68; G01N 33/566; C40B 30/04; A61K 38/16; A61K 31/7088; C07K 14/00; C07K 16/00; C07H 21/00; A01K 67/00

(52) U.S. Cl. 424/130.1; 435/6; 436/501; 506/9; 514/12; 514/44 R; 530/350; 530/387.1; 536/23.1; 800/8

(57) ABSTRACT

The invention provides novel recombinant Gβ₅ complex proteins, novel methods of identifying compounds that modulate the conformation of Gβ₅ complex, novel methods of treating disorders, including neurological and metabolic disorders, with modulators of Gβ₅ complex activity, and a mouse model of obesity, where the expression level of Gβ₅ complex is reduced by targeted deletion of one allele of a gene encoding a member of the Gβ₅ complex.

### RELATED APPLICATIONS

This application is a continuation-in-part of PCT patent application PCT/US2008/000819 filed on Jan. 23, 2008, which claims priority under 35 USC §119 to U.S. Application No. 60/881,847 filed Jan. 23, 2007, the disclosure of which is incorporated by reference in its entirety.

### GOVERNMENT SUPPORT

Work described herein may have been supported in part by NIH Grant number GM060019. The U.S. Government may have certain rights in the invention.

### FIELD OF THE INVENTION

The invention is directed generally to the fields of molecular biology, biophysics and biochemistry. More particularly, the invention provides novel recombinant Gβ₅ complex proteins, novel methods of identifying compounds that modulate the conformation of Gβ₅ complex, novel methods of treating disorders, including neurological disorders and metabolic disorders (obesity), with modulators of Gβ₅ complex activity, and a mouse model of obesity, where the expression level of Gβ₅ complex is reduced by targeted deletion of one allele of the genes encoding the members of the Gβ₅ complex.

### BACKGROUND OF THE INVENTION

G protein mediated signaling represents a major mode of signal transduction in eukaryotic cells. A signal is initiated with the binding of an agonist to heptahelical G-protein coupled receptors (GPCRs) at the plasma membrane. This activates the heterotrimeric G protein coupled to the receptor through the exchange of GDP for GTP on the Gα subunit and its subsequent dissociation from Gβγ. The dissociated subunits bind to downstream effectors until GTP hydrolysis results in the return of the Gα subunit to the GDP-bound state. GPCRs exert their effects by regulating ion channels, second messenger production, and protein kinase cascades, which in turn control neuronal activity, gene expression, plasticity, differentiation, morphogenesis, and migration. Regulators of G protein Signaling (RGS) constitute a diverse family of proteins which modulate this signaling cascade in many ways (1, 2).

Gβ₅-RGS-R7BP, hereinafter “Gβ₅ complex,” is a neuronal protein complex known to regulate signal transduction through heterotrimeric G proteins. Gβ₅ complex exists as a heterotrimer involving three polypeptide chains: (i) Gβ₅, which is a member of the family of G protein β subunits, (ii) a representative of RGS7 family, and (iii) R7S7-binding protein (R7BP), which is known as the membrane anchor of the complex. However, the Gβ₅-RGS complexes can exist in vivo as both dimers, which are cytosolic and trimers with R7BP, a membrane-bound form (3). The RGS7 family of proteins (R7 family) is comprised of RGS6, 7, 9 and 11, all of which contain the C terminal RGS box, a DEP (Dishevelled, Egl-10, Pleckstrin) domain localized in the N-terminal part of the molecule, and a centrally positioned GGL (G-gamma-like) domain. In both vertebrates and invertebrates, members of this RGS family have only been found in the central nervous system (4, 5). It appears that each family member is expressed in distinct regions of rodent brain, with RGS7 being the most abundant and widely distributed (6).

A distinctive feature of R7 family RGS proteins is that they exist as stably associated heterodimers with Gβ₅; neither R7RGS proteins nor Gβ₅ have been found apart from each other in native tissues (7-9). Similar to the R7 family RGSs, Gβ₅ has been detected only in neuronal tissues and cells (10-12). Gβ₅-RGS association requires the presence of the GGL domain, which binds with high affinity to Gβ₅ but not to other Gβ subunits (13-15). While the functional role of Gβ₅ in the dimer is still not clear (12, 16, 17), it has been established that Gβ5-RGS association is necessary for the stabilization of the heterodimer against the proteolysis of the subunits, both in reconstituted cellular systems and in animal models (8, 18-20).

Body weight control and G protein signaling. The brain receives information about the condition of energy stores in the body via insulin, leptin and cytokines released by adipocytes, and neuropeptides which originate within the gastrointestinal system, like ghrelin (42, 43). Leptin and insulin cross the blood-brain barrier and initiate events that result in decreased food intake and increased energy expenditure. There are many targets for insulin and leptin in the brain. Perhaps the most important site is the hypothalamus, where they activate or inhibit certain neuron populations (23), for example, neurons responsible for the production of a crucial catabolic pro-peptide, 32 kDa pro-opiomelanocortin (POMC). Ghrelin, a peptide released from gastrointestinal tract (24) stimulates hypothalamic neurons producing agouti-related protein (AGRP) and Neuropeptide Y (NPY). The receptors of POMC products, NPY, ghrelin and AGRP belong to the family of G protein coupled receptors, GPCRs.

### SUMMARY OF THE INVENTION

The invention provides methods of identifying a compound capable of modulating the conformation of Gβ₅ complex. The methods may comprise determining the interaction between a first Gβ₅ complex fusion protein and a second Gβ₅ complex fusion protein and comparing the determined interaction in the presence and absence of a test compound, such that a difference in the determined interaction identifies that the compound is capable of modulating the conformation of the Gβ₅ complex.

In some embodiments, a proximity-based assay is used to determine the interaction between the first and second Gβ₅ complex proteins. For example, a proximity-based assay may be a FRET-based or BRET-based assay. In other embodiments, an affinity-based assay is used to determine the interaction between the first and second Gβ₅ complex proteins. In further embodiments, a functional assay is used to determine the interaction between the first and second Gβ₅ complex proteins. One such functional assay measures a signal transduction event.

At least one of the Gβ₅ complex fusion proteins may comprise an RGS protein. The RGS protein may be any protein capable of association with Gβ₅. In some embodiments, the RGS protein is selected from the group consisting of RGS6, RGS7, RGS9, RGS11, or a homolog, chimeric protein, or derivative of any of them. In some embodiments, a mutated form of these RGS proteins may be used. At least one of the Gβ₅ complex fusion proteins may comprise a Gβ₅ subunit or a derivative thereof, such as a mutant with certain properties such as an altered affinity for an RGS protein.

An exemplary method of the invention comprises expressing in a host cell a first hybrid DNA sequence encoding a fusion protein comprising an RGS protein and a fluorescence acceptor or donor, and a second hybrid DNA sequence encoding a fusion protein comprising a G protein subunit and a fluorescence acceptor or donor; contacting the host cell with a test compound; exciting the fluorescence donor at a particular wavelength; detecting fluorescence emission of the acceptor; and comparing the fluorescence emission in the presence and absence of the test compound, such that a difference in fluorescence emission identifies the compound as capable of modulating the conformation of Gβ₅ complex.

The invention further provides high throughput methods of identifying a compound capable of modulating the conformation of Gβ₅ complex. In some embodiments, the identified compound may be selected from a library of compounds, such as chemicals or small molecules.

The invention also provides compounds identified as capable of modulating the conformation of Gβ₅ complex. In some embodiments, an identified compound induces the open conformation of Gβ₅ complex, and in other embodiments, an identified compound induces the closed conformation of Gβ₅ complex. In some embodiments, an identified compound is an agonist of Gβ₅ complex activity, and in other embodiments, an identified compound is an antagonist of Gβ₅ complex activity. The Gβ₅ complex activity may be associated with a disorder or disease, such as a neurological disorder or obesity.

Provided by the invention are pharmaceutical compositions comprising compounds identified as capable of modulating the conformation of Gβ₅ complex. The compositions are effective for the treatment of disorders associated with Gβ₅ complex activity, such as a neurological disorder or obesity.

Also provided by the invention are recombinant proteins comprising Gβ₅ complex in an open conformation. For example, recombinant proteins comprising a mutation of the Gβ₅ binding site in the DEP domain are provided. Another example is a mutation of the DEP domain binding site in the Gβ₅ molecule.

Another aspect of the invention provides methods of identifying a compound capable of modulating weight gain. The methods may comprise administering a test compound to a first mouse comprising a deletion of one allele of a gene encoding a Gβ₅ protein, and comparing weight gain of the first mouse to the weight gain of a second mouse comprising the deletion of one allele of a gene encoding a Gβ₅ protein not administered the test compound, such that a difference in weight gain between the first mouse and the second mouse identifies that the test compound is capable of modulating weight gain. In some embodiments, the first mouse and the second mouse comprising a deletion of one allele of a gene encoding a Gβ₅ protein are Gβ₅ heterozygous mice. In other embodiments, the first mouse and the second mouse comprising a deletion of one allele of a gene encoding a Gβ₅ protein are RGS7 heterozygous mice.

A further aspect of the invention provides methods for predicting the onset of obesity in an individual. The methods may comprise identifying a mutation in a Gβ₅ gene and correlating the identified mutation with a prediction of the onset of obesity in an individual carrying such a mutation.

Yet another aspect of the invention provides a mouse model of obesity, in which the expression level of Gβ₅ complex is reduced by targeted deletion of one allele of a gene encoding a member of the Gβ₅ complex.

Yet further provided by the invention are kits for identifying a compound capable of modulating the conformation of the Gβ₅ complex. The kits may comprise DNA constructs encoding Gβ₅ complex fusion proteins and a host cell for transfection with the DNA constructs.

### BRIEF DESCRIPTION OF THE FIGURES

FIG. 1 shows the interaction of DEP domain of RGS7 with native Gβ₅-RGS complexes. GST fusions of DEP domains of RGS7 or RGS9 (R7-DEP, R9-DEP) or GST were immobilized on Glutathione Sepharose beads. The beads were incubated batch-wise with extracts from mouse brain or bovine photoreceptor outer segments (OS), as described in Example 1. After the slurry was spun down, and the unbound material was collected, the resin was washed and eluted with SDS-PAGE Sample Buffer. The unbound (U) and eluted (E) material was analyzed by western blot. A. Mouse brain or bovine OS extracts were subjected to pull-down with GST fusion of R7-DEP, using GST as the negative control. The fractions from the pull-down assay were probed with the antibodies to RGS7 or RGS9, respectively. B. Mouse brain extract was subjected to pull-down and the fractions were probed for the presence of Gβ subunits Gβ₅ and Gβ₁. C. DEP domains of RGS7 and RGS9 were compared in their ability to bind Gβ₅ complexes in brain and OS extracts. D. The amount of Gβ₅-RGS7 bound to the GST-R7DEP beads determined as a fraction of total Gβ₅-RGS7 in the brain extract. Gels were scanned and analyzed with Scion software. To ensure that the amount of Gβ₅ loaded in the unbound and eluted lanes was within the linear range of the film and scanner, material in the eluate was 5 times more concentrated relative to the unbound. Data show the mean±standard deviation from eight independent experiments.

FIG. 2 shows the interaction of RGS7 DEP domain with recombinant Gβ₅ or Gβ₁. A. Gβ₅ or the Gα subunits Gαᵢ and Gα_{q} were translated in vitro in the presence of [³⁵S]-methionine, and subjected to pull-down assay with the GST fusion of RGS7 DEP domain (R7-DEP). The unbound (U) and eluted (E) material was resolved by SDS-PAGE, transferred to nitrocellulose and detected by autoradiography. B. Left panel: Western blot (anti-Gβ₅ antibody) of the unbound (U) and eluted (E) fractions from the R7-DEP GST pull-down of Gβ_{5γ2} complex transiently expressed in HEK 293 cells. Right panel: pull-down with the DEP domains of RGS7 and RGS9 of transiently expressed Gβ_{1γ2} complex; the fractions probed with anti-Gβ₁ antibody. C. Homogeneous baculovirus-expressed Gβ_{1γ2} complex was subjected to the pull-down with R7-DEP and the fractions were analyzed by western blot using anti-Gβ₁ antibody.

FIG. 3 shows that endogenous DEP domain reduces the interaction of RGS7-Gβ₅ dimers with the recombinant RGS7 DEP. A. COS-7 cells were transfected with full-length RGS7, ΔDEP-RGS7, and RGS7²⁴⁹⁻⁴⁶⁹. The schematic drawings of these constructs depict the approximate location of the DEP (diamond), GGL (grey rectangle) and RGS (black rectangle) domains along the RGS7 polypeptide (black line). All the constructs were co-transfected together with Gβ₅ cDNA to ensure their stability. Total cell lysates were prepared 48 hours post-transfection and incubated with GST-R7DEP or GST bound to Glutathione Sepharose beads. Unbound (U) and eluted (E) fractions were analyzed by SDS PAGE and detected after western blotting. The filters were first probed with the antibody against RGS7, developed, and subsequently probed with the antibody against Gβ₅. Since each antibody detected a single band, and the antigens differed significantly in molecular weight, stripping of the blots between the probing with the two antibodies was not required. B. The amount of the DEP-less Gβ₅-RGS7 constructs bound to the GST-R7DEP beads compared to that of the full-length Gβ₅-RGS7. Gels were scanned and analyzed with Scion software as described in the legend to FIG. 1D, and in more detail in Example 1. Data show the mean±standard deviation from three (DEP-less constructs) and four (full-length RGS7) independent experiments. C. As in A, cells were transfected with either RGS7, ΔDEP-RGS7, or RGS7²⁴⁹⁻⁴⁶⁹ constructs together with Gβ₅. In addition, the transfection mixture contained the plasmid encoding the fusion of yellow fluorescent protein (YFP) with the first 248 amino acids of RGS7, YFP-RGS7¹⁻²⁴⁸. Cell lysates prepared as in (A) were subjected to immunoprecipitation with an antibody against the C-terminus of RGS7 bound to Protein A Sepharose. The unbound (U) and eluted (E) fractions were analyzed by western blotting with anti-GFP antibody to detect the YFP-RGS7¹⁻²⁴⁸ fusion protein.

FIG. 4 shows that fluorescence resonance energy transfer from CFP-Gβ₅ to YFP-RGS7 is reduced by the GST fusion of RGS7 DEP domain. To measure FRET, COS-7 cells were transiently transfected with plasmids encoding YFP-RGS7 and CFP-Gβ₅. Control cells were co-transfected with YFP-RGS7 together with untagged Gβ₅, and CFP-Gβ₅ together with untagged RGS7. The lysates of these cells were subjected to spectroscopic analysis, as described in Example 1. Shown are the resulting FRET spectra obtained after subtraction of the YFP background fluorescence during the CFP excitation and CFP “bleed-through” into the YFP emission channel. A. Right panel: GST R7-DEP (black symbols) or buffer (open symbols) was added to the lysate prior to recording of the spectra. Left panel: GST (black symbols) or buffer (white symbols) was added to an aliquot of the same lysate. B. Western blot showing the expression of the fluorescent proteins in COS-7 lysate (15 μg of total protein) that was used in these experiments. C. Our model: the drawing illustrates YFP fused to the N-terminus of RGS7 and CFP fused to the N-terminus of Gβ₅, which form a strong FRET pair. This state represents a “closed” conformation of the Gβ₅-RGS7 heterodimer. In the presence of GST-R7DEP fusion (black circle depicts GST), the intrinsic DEP domain cannot re-associate with Gβ₅, but since the YFP fluorophore remains sufficiently close to the CFP tag, FRET can still occur, albeit to a lesser degree. This state represents an “open” conformation of the Gβ₅-RGS7 molecule.

FIG. 5 shows that DEP-Gβ₅ interaction within the Gβ₅-RGS7 dimer is dynamic. COS-7 cells were transiently transfected with YFP fusion of full-length RGS7 (YFP-RGS7) and CFP-Gβ₅. Three 2 ml aliquots of the cell lysate were analyzed using a fluorescence spectrophotometer. To one portion of the lysate was added purified GST-R7DEP (black diamonds), another aliquot was mixed with GST (black squares) and the third aliquot was mixed with buffer (white squares). The protein stocks (65 μM) or buffer were added to the quartz cuvette in 50 μl increments with continuous stirring. After each addition, total fluorescence values at YFP maximum (525 nm, shown in A) and CFP maximum (490 nm, B) were determined; the excitation wavelength was set at 433 nm, and the spectra were taken as described in Materials and Methods. A. Raw data showing a single representative experiment where 525 nm fluorescence is plotted against the concentration of GST or GST-R7DEP in the mixture. The specific effect of GST-R7DEP is clearly discernable compared to the effect of lysate dilution, which is seen as the stable decline of the signal in the presence of buffer or GST. B. Total 490 nm (CFP emission maximum) fluorescence recorded in the same experiment; raw data. C. Summary of data from four independent experiments (independent COS-7 cell transfections). Y axis: Data show the difference between fluorescence recorded in the presence of GST-R7DEP (F_{DEP}) and fluorescence measured in the presence of GST (F_{GST}), F_{DEP}−F_{GST}, mean±standard deviation. Black symbols designate YFP fluorescence; white, CFP. Note that the F_{DEP}−F_{GST} difference is negative for YFP and positive for CFP. The lines connecting the values represent linear regression fit of the data (r²>0.94 for both YFP and CFP).

FIG. 6 shows the potential Gβ₅ binding site on RGS7 DEP. A. Amino acid sequence alignment of putative DEP domains of R7RGS proteins. Multiple sequence alignment was generated by MAFFT using iterative refinement method and JTT200 scoring matrix (25, 26) and ESPript 2.2 (27). Identical residues are shaded in red. Conserved blocks of amino acids are represented as blue boxes. Abbreviations: hu, human; by, bovine; ce, Caenorhabditis elegans; sc, Saccharomyces cerevisiae. The arrow indicates the position of the two acidic residues, Glu73 and Asp74 of RGS7 that were mutated. B. Commassie-stained gel illustrating the purity of the GST fusion proteins: GST-R7DEP, GST-R9DEP and the mutant of GST-R7DEP (ED/SG), where the amino acids Glu73 and Asp74 in RGS7 were substituted by Ser and Gly, respectively. Equal amounts of these proteins were loaded on the glutathione beads in the subsequent pull-down assays. C. The ED/SG mutant was tested for its ability to bind transiently expressed R7BP (FLAG antibody). D. Left panel: representative experiment where the Gβ₅-RGS7²⁴⁹⁻⁴⁶⁹ complex was transiently expressed in COS-7 and subjected to the pull-down with the indicated GST fusion proteins. The unbound (U) and eluted (E) fractions were analyzed by western blot with the Gβ₅ antibody. Right panels: mouse brain extract was subjected to the pull-down and the fractions were probed with either anti-RGS7 or anti-Gβ₅ antibodies.

FIG. 7 shows the effect of the ED/SG mutation and R7BP on the function of Gβ₅-RGS7 complex. The double mutation E73S/D74G was introduced into the full-length RGS7 in the pcDNA3 plasmid. The RGS7^{ED/SG} mutant was transiently transfected, along with Gβ₅ and muscarinic M3 receptor cDNAs into wild type CHO-K1 and CHO-R7BP cells. Carbachol-induced Ca²⁺ transients in CHO-R7BP cells. Cells were transfected with M3 receptor, Gβ₅, and either wild type RGS7 or the RGS7^{ED/SG} mutant. Cells were plated on glass coverslips, loaded with fura-2, and fluorescence was recorded in real time as described in Materials and Methods. Shown are the mean amplitude ±SD of the Ca²⁺ responses from five independent experiments. In each experiment, traces were recorded from 20 randomly selected Carbachol-responding cells. The determined average response was expressed as the percent value compared to the average response of cells transfected only with M3 receptor (in the absence of RGS7, Gβ₅ and R7BP), which was set as 100% response. Black bars represent CHO-R7BP cells. Gray bars represent CHO-K1 cells.

FIG. 8 shows that mutations in either DEP domain of RGS7 and Gβ₅ can inhibit their interaction. Wild type DEP domain (WT DEP) or its mutants were expressed in E. coli as GST-fusion proteins. A. These GST fusions were immobilized on beads and mixed with the Gβ5-RGS7²⁴⁹ complex expressed in cos-7 cells. The beads were washed and then eluted with an SDS-containing buffer. The unbound material (U) and the eluates (E) from the beads were resolved by SDS-PAGE, and the presence of Gβ₅-RGS7 (A, C) or R7BP (B) was revealed by immunoblot. B. R7BP was expressed in COS-7 cells in a FLAG-tagged form and tested for binding with the same GST-DEP constructs as in A. C. WT GST-DEP was used to pull-down complexes of wild-type (WT) Gβ₅ or its mutants. All the mutations were substitutions with Ala residues.

FIG. 9 shows the increased weight gain in Gβ₅−/+ mice. A. Plot of the weight (grams, mean±SD) of wild-type (black), Gβ₅−/− (blue) and Gβ₅−/+ mice (red) over the time (weeks). The bracket and star symbol show the (statistically significant) difference between the Gβ₅−/− and the wild-type mice at four weeks. B. Photograph comparing the WT and Gβ₅−/+ males at 9 months.

FIG. 10 shows that food consumption in Gβ₅−/+ mice is normal. Food consumed (grams per mouse per 5 days) by the wild-type (black), Gβ₅−/− (blue) and Gβ₅−/+ mice (red) plotted versus the time (weeks).

FIG. 11 shows the analysis of fat content in the wild-type (WT), heterozygote (HET, Gβ5−/+) and knockout (KO, Gβ5−/−) mice using dual energy X-ray absorptiometry (DEXA). Mice were anesthetized with ketamine and placed under the X ray device PIXImus2 (Lunar/GE Medical Systems). The total fat and percentage fat were determined using the established irradiation protocol and software as recommended by the manufacturer. Shown are the averages of the determined values (error bars: standard error); n=9, 10 and 8 for WT males from the three cohorts aged 15, 13 and 11 months. Mouse heads were excluded from the region of interest, which included the entire body down to the tail base.

FIG. 12 shows the increased BMI in Gβ5−/+ (HET) mice compared to wild-type (WT) and Gβ5−/− knockout (KO) mice. Mice were fed regular chow (Fat-10%/Carbs-70%/Protein-20%) and at the indicated ages, body length (nose-to-tail base distance) was determined on the isofluorane-anesthetized animals. BMI was calculated as weight divided by squared body length. (n=13 for KO; WT, 12; HET, 18).

FIG. 13 shows the attenuation of M3R-mediated Ca²⁺ response by Gβ5-RGS7. CHO cells were transiently transfected with muscarinic M3 receptor, Gβ5 and RGS7 cDNAs and grown on glass coverslips. After loading with the ratiometric Ca²⁺ chelator dye fura-2, the coverslips were imaged in real time, using the MetaFluor software. After 30 sec of perfusion with buffer, the flow is switched to the solution of 1 μM carbachol (Cch). Fluorescence was recorded from the entire field of view (40× magnification lens) containing 50-60 cells. The fluorescence values are recalculated to the concentration of free Ca²⁺ using a standard calibration procedure. Transfection efficiency in these experiments is about 80%. Other relevant technical details were described earlier ((21)). Untransfected CHO cells do not contain endogenous Gq-coupled muscarinic receptors and do not respond to acetylcholine of Cch. A. Example of Ca²⁺ traces obtained from cells expressing M3R alone (plus LacZ cDNA plasmid) and M3R in the presence of Gβ5-RGS7. B. Maximum amplitude of the responses was compared at different carbachol concentrations.

FIG. 14 shows that Gβ5-RGS7 has no effect on M1R and other Gq-coupled GPCRs. The experiment was performed on CHO cells as in FIG. 4, except other GPCRs were co-transfected instead of M3R. A. Dose-dependence from carbachol in cells expressing M1R performed as in FIG. 4B (example of raw traces is show in Appendix 1). B. Cells were transfected with the indicated GPCRs-M5R, histamine, 5HT2c and GNRH, and the ratio of maximum amplitude of Ca²⁺ response was measured in cells expressing the indicated receptor together with Gβ5-RGS7 relative to alone, at two different agonist concentrations (grey and black bars).

FIG. 15 shows that activation of muscarinic acetylcholine receptor type 3 (M3R) influences subcellular localization of Gβ5-RGS7. CHO cells were co-transfected with plasmids encoding Gβ5, YFP-RGS7 fusion protein, and wild-type human M3 or M1 muscarinic receptors. Cells were grown on glass coverslips for 24 h, and then stimulated with 100 μM carbachol (CCH) for 5 min. Cells were then fixed with paraformaldehyde and imaged in a confocal microscope to visualize YFP. Shown are representative images of single optical planes; more than ten independent experiments (time of stimulation varied 5-45 min).

FIG. 16 shows that AR compound inhibits GST-DEP interaction with Gβ5-R7²⁴⁹ complex. The GST fusion of the DEP domain of RGS7 was expressed in E. coli. The complex of Gβ5 with the C-terminal portion of the RGS7 molecule that lacks the DEP domain (R7²⁴⁹) was expressed in COS-7 cells. The pull-down of Gβ5-R7²⁴⁹ was performed using the immobilized GST-DEP, as described in FIG. 8 in the presence of indicated concentrations of AR. A. Representative western blot of the fractions. B. Quantification of the combined data from three independent experiments. Following the ECL detection, the film was scanned; the amount of signal in eluted fractions (arbitrary units) was plotted against the AR concentration used in the pull-down.

### DETAILED DESCRIPTION OF THE INVENTION

For the purposes of promoting an understanding of the principles of the invention, reference will now be made to specific embodiment and specific language will be used to describe the same. It will nevertheless be understood that no limitation of the scope of the invention is thereby intended, such alteration and further modifications of the invention, and such further applications of the principles of the invention as illustrated herein, being contemplated as would normally occur to one skilled in the art to which the invention relates.

All terms as used herein and not specifically defined herein are defined according to the ordinary meanings they have acquired in the art. Such definitions can be found in any technical dictionary or reference known to the skilled artisan, such as the McGraw-Hill Dictionary of Scientific and Technical Terms (McGraw-Hill, Inc.), Molecular Cloning: A Laboratory Manual (Cold Springs Harbor, N.Y.), and Remington's Pharmaceutical Sciences (Mack Publishing, PA). These references, along with those references and patents cited herein are hereby incorporated by reference in their entirety.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “test substance”, “test compound”, “candidate therapeutic agent” “agonist”, “antagonist” or “agent” are used interchangeably herein, and the terms are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, and the like. A test substance can be assayed in accordance with the methods of the invention at any stage during clinical trials, during pre-trial testing, or following FDA-approval.

As used herein, the term “gene” or “polynucleotide” means the gene and all currently known variants thereof and any further variants which may be elucidated, including different species.

“Variant” polynucleotides and polypeptides include molecules containing one or more deletions, insertions and/or substitutions compared to the nucleic acids. Variant polynucleotides can encode the same or a functionally-equivalent polypeptide. The term “variant” when used in context of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

“Derivative” polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

“Detect” refers to identifying the presence, absence or amount of the object to be detected.

As used herein, the term “animal”, “individual', “subject” or “patient” is meant to include, for example, humans, sheep, elks, deer, mule deer, minks, mammals, monkeys, horses, cattle, pigs, goats, dogs, cats, rats, mice, birds, chicken, reptiles, fish, insects and arachnids. Preferred subjects are humans.

“Mammal” covers warm blooded mammals that are typically under medical care (e.g., humans and domesticated animals). Examples include feline, canine, equine, bovine, and human, as well as just human.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

The term “sample” is meant to be interpreted in its broadest sense. A “sample” refers to a biological sample, such as, for example; one or more cells, tissues, or fluids (including, without limitation, plasma, serum, whole blood, cerebrospinal fluid, lymph, tears, urine, saliva, milk, pus, and tissue exudates and secretions) isolated from an individual or from cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

Numerous well known tissue or fluid collection methods can be utilized to collect the biological sample from the subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

“Microarray” is an array of distinct polynucleotides, oligonucleotides, polypeptides, peptides, or antibodies affixed to a substrate, such as paper, nylon, or other type of membrane; filter; chip; glass slide; or any other type of suitable support.

### Compositions:

There is a need for novel compounds to regulate the function of neuronal cells. There is also a need for novel compounds for treating diseases and disorders associated with Gβ₅ complex activity, such as the apparent deregulation of body weight. The invention is directed to these and other ends.

The invention is based on the discovery of a dynamic intra-molecular interaction within the Gβ₅ complex that has a strong effect on the activity of the complex. Gβ₅ complex is known to exist as a heterotrimer involving three polypeptide chains: Gβ₅, an RGS7 protein, and an RGS7 binding protein (R7BP). RGS7 proteins are known to consist of three domains: RGS domain, which is responsible for interaction with G proteins; GGL domain, which irreversibly binds to Gβ₅; and DEP domain, which binds to R7BP. The inventors have surprisingly discovered that the DEP domain of the RGS protein directly interacts with Gβ₅, leading to at least two different conformations that represent different states of biological activity of the Gβ₅ complex. When the DEP domain is bound to the Gβ₅ subunit, the Gβ₅ complex is in a “closed conformation” and the molecule is inactive. When the DEP domain is dissociated from the Gβ₅ subunit, the Gβ₅ complex is in an “open conformation” and the molecule is active.

An aspect of the invention provides recombinant proteins comprising Gβ₅ complex in an open conformation. In one illustrative embodiment, recombinant proteins comprising a mutation of the Gβ₅ binding site in the DEP domain are provided. In another illustrative embodiment, a mutation of the DEP domain binding site in the Gβ₅ molecule is provided.

Another aspect of the invention provides novel compounds that are capable of modulating Gβ₅ complex conformation and thereby inducing a change in the activity of Gβ₅ complex. In some embodiments, the novel compounds induce at least one of either a closed or an open conformation of the molecule. In other embodiments, the novel compounds are capable of inducing a more open conformation, and therefore more active Gβ₅ complex, and in yet other embodiments, the novel compounds are capable of inducing a more closed conformation, and therefore less active Gβ₅ complex.

### Assays:

The invention also provides novel methods and kits for identifying these compounds. These methods may comprise determining the interaction between a first Gβ₅ complex fusion protein and a second Gβ₅ complex fusion protein and comparing the determined interaction in the presence and absence of a test compound, wherein a difference in the determined interaction identifies that the compound is capable of modulating the conformation of Gβ₅ complex. The kits may comprise DNA constructs encoding the Gβ₅ complex fusion proteins, a host cell for transfection with the DNA constructs, instructions for use of the kit, and a container to hold the components of the kit.

The methods of the invention may be automated for high capacity-high throughput screening (HTS) in which large numbers of compounds may be tested to identify compounds with the desired activity. In some embodiments of the invention, the test compounds to be screened for ability to modulate Gβ₅ complex activity may be members of a library of test compounds. Such a library of test compounds may include peptides mimicking natural binding partners of Gβ₅ complex, such as G protein subunits, and other members of this pathway. The library may also contain molecules identified through computer modeling or other designed molecules. The library may also be a commercially available library of small molecules. The high throughput methods of the invention may be adapted to screen cDNA libraries for expressed proteins that modulate Gβ₅ complex activity.

Before carrying out the assays, it may be necessary to clone, express, and, in some of the assays, purify the Gβ₅ fusion proteins. Recombinant expression methods are well known to the skilled artisan and are described, for example, in Sambrook et al., Molecular Cloning: A Laboratory Manual, Cold Springs Harbor Laboratory, 3rd ed., Cold Springs Harbor, N.Y. (2001). Other references describing molecular biology and recombinant DNA techniques include, for example, DNA Cloning 1: Core Techniques, (D. N. Glover, et al., eds., IRL Press, 1995); DNA Cloning 2: Expression Systems, (B. D. Hames, et al., eds., IRL Press, 1995); DNA Cloning 3: A Practical Approach, (D. N. Glover, et al., eds., IRL Press, 1995); DNA Cloning 4: Mammalian Systems, (D. N. Glover, et al., eds., IRL Press, 1995); Oligonucleotide Synthesis (M. J. Gait, ed., IRL Press, 1992); Nucleic Acid Hybridization: A Practical Approach, (S. J. Higgins and B. D. Hames, eds., IRL Press, 1991); Transcription and Translation: A Practical Approach, (S. J. Higgins & B. D. Hames, eds., IRL Press, 1996); R. I. Freshney, Culture of Animal Cells: A Manual of Basic Technique, 4^{th} Edition (Wiley-Liss, 1986); and B. Perbal, A Practical Guide To Molecular Cloning, 2^{nd} Edition, (John Wiley & Sons, 1988); and Current Protocols in Molecular Biology (Ausubel et al., eds., John Wiley & Sons), which is regularly and periodically updated.

Suitable vectors for expression of DNA constructs may include, for example, bacterial or yeast plasmids, wide host range plasmids and vectors derived from combinations of plasmid and phage or virus DNA. Vectors derived from chromosomal DNA are also included. Furthermore, an origin of replication and/or a dominant selection marker may be present in the vector according to the invention. The vectors according to the invention are suitable for transforming, transfecting, or infecting a host cell.

DNA constructs may be expressed in any cells suitable for use as host cells for recombinant DNA expression, including any eukaryotic or prokaryotic host cells. Thus, a host cell which comprises the DNA or expression vectors according to the invention is also within the scope of the invention. Suitable host cells transformed with the DNA constructs may be fermented and subjected to conditions which facilitate the expression of the heterologous DNA, leading to the formation of large quantities of the desired protein. Non-limiting examples of preferred host cells suitable for protein expression in accordance with the invention include bacterial, CHO, and COS cells.

Selection of an appropriate purification procedure for the chimeric polypeptides present in the host cell extract or culture medium is routine to one skilled in the art, and may be based on the properties of the polypeptides, such a size, charge and function. Methods of purification include centrifugation, electrophoresis, chromatography, dialysis or a combination thereof. As known in the art, electrophoresis may be utilized to separate the proteins in the sample based on size and charge. Electrophoretic procedures are well known to the skilled artisan, and include isoelectric focusing, sodium dodecyl sulfate polyacrylamide gel electrophoresis (SDS-PAGE), agarose gel electrophoresis, and other known methods of electrophoresis.

The purification step may be accomplished by a chromatographic fractionation technique, including size fractionation, fractionation by charge and fractionation by other properties of the polypeptides being separated. As known in the art, chromatographic systems include a stationary phase and a mobile phase, and the separation is based upon the interaction of the polypeptides to be separated with the different phases. In some forms of the invention, column chromatographic procedures may be utilized. Such procedures include partition chromatography, adsorption chromatography, size-exclusion chromatography, ion-exchange chromatography and affinity chromatography. An affinity tag may also be engineered into the desired polypeptide for purification purposes. For example, the DNA constructs of the invention may encode glutathione S-transferase (GST) to facilitate protein purification on glutathione sepharose beads.

At least one Gβ₅ complex fusion protein may be a G protein signaling regulator protein (RGS protein) capable of associating with Gβ₅. Exemplary RGS proteins suitable for the invention include any member of the R7 family of RGS proteins, including but not limited to RGS6, 7, 9 and 11, or any homolog, chimeric protein, or derivative of an RGS protein. In some embodiments, a mutated form of an RGS protein is used. At least one Gβ₅ complex fusion protein may be a Gβ₅ protein subunit, including any portion of the subunit capable of associating with an RGS protein, or a derivative of a Gβ₅ protein subunit, such as a mutant with properties such as an altered affinity for an RGS protein. A derivative form of an RGS protein or a Gβ₅ protein subunit may be arrived at by modification of the native amino acid sequence by such modifications as insertion, substitution or deletion of one or more amino acids, or it may be a naturally occurring derivative.

This invention further contemplates a method of generating sets of combinatorial mutants of different proteins, e.g. RGS protein, Gβ₅ protein subunit; as well as truncation mutants, and fusion proteins thereof, and is especially useful for identifying potential variant sequences (e.g. homologs). Such proteins, when expressed from recombinant DNA constructs, can be used in gene therapy protocols. Likewise, mutagenesis can give rise to homologs which have intracellular half-lives dramatically different than the corresponding wild-type protein.

Also provided by the invention are chemically modified derivatives of the peptides and polypeptides of the invention that may provide additional advantages such as increased solubility, stability, and circulating time of the polypeptide. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

In addition, amino acid sequence variants of the present invention include, but are not limited to, variants that share at least 40%, 50%, 60%, 61%, 67%, 70%, 74%, 76%, 80%, 81%, 84%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% nucleotide sequence identity with RGS, M3R Gβ₅ subunit, or any other protein or polypeptide that a user may select.

Polypeptide and peptide variants include variants differing by the addition, deletion, or substitution of one or more amino acid residues. For example, to isolate RGS7 polypeptides or peptides, it may be useful to encode a tagged RGS7 peptide or polypeptide that can be recognized by a commercially available antibody. In particular, a peptide or polypeptide can be fused or linked to epitope tags (e.g., FLAG, HA, GST, thioredoxin, maltose binding protein, etc.), or affinity tags such as biotin and/or streptavidin. As one example, a system for the ready purification of non-denatured fusion proteins expressed in human cell lines has been described by Janknecht et al., (1991, Proc. Natl. Acad. Sci. USA, 88:8972-8976). In this system, the gene of interest is subcloned into a vaccinia recombination plasmid such that the open reading frame of the gene is translationally fused to an amino-terminal tag having six histidine residues. The tag serves as a matrix-binding domain for the fusion protein. Extracts from cells infected with the recombinant vaccinia virus are loaded onto an Ni²⁺ nitriloacetic acid-agarose column and histidine-tagged proteins are selectively eluted with imidazole-containing buffers.

A peptide or polypeptide tagged with an epitope or protein may also be engineered to contain a cleavage site located between the binder coding sequence and the tag coding sequence. This can be used to remove the tag, and isolate the RGS7 peptide or polypeptide. The RGS7 peptides or polypeptides of the invention can be covalently attached to chemical moieties via the amino acid backbone. For these purposes, the peptides or polypeptides may be modified by N- or C-terminal processing of the sequences (e.g., proteolytic processing), deletion of the N-terminal methionine residue, etc. The polypeptides may also be modified with a detectable label, such as an enzymatic, fluorescent, isotopic or affinity label to allow for detection and isolation of the protein, as described in detail herein.

Also included are modified polypeptides and peptides in which one or more residues are modified, and mutants comprising one or more modified residues. Amino acid variants of the invention can be generated by employing the techniques of gene-shuffling, motif-shuffling, exon-shuffling, and/or codon-shuffling (collectively referred to as “DNA shuffling”). DNA shuffling can be employed to generate peptides or polypeptides with altered activity. See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol., 16(2):76-82; Hansson, et al., 1999, J. Mol. Biol., 287:265-76; and Lorenzo and Blasco, 1998, Biotechniques, 24(2):308-313, the contents of each of which are hereby incorporated by reference in its entirety.

Polypeptides or peptides may also be modified with a label capable of providing a detectable signal, either directly or indirectly, including, but not limited to, radioisotope, fluorescent, and enzyme labels. Fluorescent labels include, for example, Coumarin (e.g., Hydroxycoumarin, Aminocoumarin, Methoxycoumarin), R-Phycoerythrin (PE), Fluorescein, FITC, Fluor X, DTAF, Auramine, Alexa (e.g., ALEXA FLUOR™ 350, -430, -488, -532, -546, -555, -568, -594, -633, -647, -660, -680, -700, -750), BODIPY-FL, Sulforhodamine (e.g., Texas Red™), Carbocyanine (e.g., Cy2, Cy3, Cy3.5, Cy5, Cy5.5, Cy7), Rhodamine, XRITC, TRITC, Lissamine Rhodamine B, Peridinin Chlorphyll Protein (PerCP), Allophycocyanin (APC), PE-Cy5 conjugates (e.g., Cychrome, TRI-COLOR™, QUANTUM RED™), PE-Cy5.5 conjugates, PE-Cy7 conjugates, PE-Texas Red conjugates (e.g., Red613), PC5-PE-Cy5 conjugates, PerCP-Cy5.5 conjugates (e.g., TruRed), APC-Cy5.5 conjugates, APC-Cy7 conjugates, ECD-PE-Texas Red conjugates, Sulfonated Pyrene (e.g., Cascade Blue), AMCA Blue, Lucifer Yellow.

Preferred isotope labels include ³H, ¹⁴C, ³²P, ³⁵S, ³⁶Cl, ⁵¹Cr, ⁵⁷Co, ⁵⁹Fe, ⁹⁰Y, ¹²⁵I, ¹³¹I, and ¹⁸⁶Re. Preferred enzyme labels include peroxidase, β-glucuronidase, β-D-glucosidase, β-D-galactosidase, urease, glucose oxidase plus peroxidase, and alkaline phosphatase (see, e.g., U.S. Pat. Nos. 3,654,090; 3,850,752 and 4,016,043). Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, colloidal gold (e.g., NANOGOLD™), Tyramide Signal Amplification (TSA™), are known in the art, and are commercially available (see, e.g., ABC kit, Vector Laboratories, Inc., Burlingame, Calif.; NEN™ Life Science Products, Inc., Boston, Mass.; Nanoprobes, Inc., 95 Horse Block Road, Yaphank, N.Y.).

In some embodiments, the methods of the invention employ proximity-based assays to determine the interaction between the first and second Gβ₅ complex fusion proteins. Assays used to determine the proximity between two binding partners are well known to the skilled artisan and may be easily modified to identify Gβ₅ complex modulating compounds in accordance with the invention.

Some proximity-based assays useful for the invention include assays based on the transfer of energy from one binding partner to the other binding partner. In some embodiments, the identifying methods utilize a fluorescence resonance energy transfer (FRET) assay, wherein fluorescence energy is transferred from one binding partner to the other, to detect the interaction between two Gβ₅ protein complex components. In other embodiments, the identifying methods utilize a bioluminescence resonance energy transfer (BRET) assay, wherein energy from luciferase is transferred to a fluorescent protein, to detect the interaction between the two Gβ₅ protein complex components.

An exemplary FRET screening method involves expressing in a host cell a first hybrid DNA sequence encoding a fusion protein comprising a G protein signaling regulator protein and a fluorescence acceptor or fluorescence donor, and a second hybrid DNA sequence encoding a fusion protein comprising a G protein subunit and a fluorescence acceptor or fluorescence donor; contacting the host cell with a test compound; exciting the fluorescence donor at a particular wavelength; detecting fluorescence emission of the acceptor, and comparing the emission in the presence and in the absence of the compound. A difference in fluorescence emission as a result of the presence of the test compound indicates that the compound is capable of modulating the conformation of Gβ₅ complex. In some embodiments, a test compound may be a Gβ₅ complex agonist that induces an open conformation of Gβ₅ complex, leading to reduced FRET interaction between the RGS and G protein subunit fusion proteins as compared to the FRET measured in the absence of the test compound. In other embodiments, a test compound may be a Gβ₅ complex antagonist that induces a closed conformation of Gβ₅ complex, leading to increased FRET interaction between the RGS and G protein subunit fusion proteins as compared to the FRET measured in the absence of the test compound.

The fluorescence donors of the invention may be any protein or molecule that may be excited at a particular wavelength to transfer energy to a fluorescence acceptor. The fluorescence acceptors of the invention may be any protein or molecule that can emit energy upon transfer of energy from a fluorescent donor. The selection of the appropriate combination of fluorescent acceptor and fluorescent donor for the methods of the invention is well within the purview of the skilled artisan. In some embodiments, the donor may be cyan fluorescent protein and the acceptor may be yellow fluorescent protein. In other embodiments, the fluorescent donor/acceptor pair may be blue fluorescent protein and green fluorescent protein. Methods for measurement of FRET activity are also known in the art.

In other embodiments, the methods of the invention employ affinity-based (“pull-down”) assays to determine the interaction between the first and second Gβ₅ complex fusion proteins. For example, a first Gβ₅ complex fusion protein may be immobilized on a solid support and incubated with a labeled form of a second Gβ₅ complex fusion protein, in the presence and absence of a test compound. The solid support may then be separated from the reaction mixture and the amount of label on the collected support may be detected. If the labeled second Gβ₅ protein interacts with the immobilized first Gβ₅ protein, the label will be detected on the collected support. If the two Gβ₅ complex fusion proteins do not interact or interact less, for example due to the presence of the test compound, the label will not be detected or will be detected less on the collected support.

Many solid supports and labeling materials are available to the skilled artisan. The solid support may be any material suitable for immobilization of one of the components of the Gβ₅ complex. For example, the solid support may be sepharose beads or the surface of an ELISA plate. The label on the second Gβ₅ complex may be any label or affinity tag suitable for detection of the bound protein. For example, the second Gβ₅ complex fusion protein may be expressed with an epitope that can be detected with an antibody. A reporter gene may be fused directly to the second Gβ₅ complex fusion protein for utilization as an affinity label. The reporter protein may be a fluorescent protein for direct detection of fluorescence, or an enzyme such as luciferase, phosphatase or peroxidase for detection of their reaction products. The second Gβ₅ complex fusion protein may also be labeled with a radioactive isotope.

In other embodiments, the methods of the invention employ functional assays to determine the interaction between the first and second Gβ₅ complex fusion proteins. For example, Gβ₅ complex is known to inhibit muscarinic receptor-mediated calcium mobilization. If the first and second Gβ₅ complex fusion proteins interact to form Gβ₅ complex, the inhibition of Ca⁺² may be detected, for example with a fluorescence indicator. Accordingly, test compounds may be added to the Gβ₅ complex to test the ability of the compounds to inhibit Gβ₅ calcium inhibiting activity, for example by inducing an closed conformation, or to stimulate Gβ₅ calcium inhibiting activity, for example by inducing an open conformation.

In a preferred embodiment, the high-throughput screening assay (HTS) screening assay is used to screen a diverse library of member compounds. The “compounds” or “candidate therapeutic agents” or “candidate agents” can be any organic, inorganic, small molecule, protein, antibody, aptamer, nucleic acid molecule, or synthetic compound.

In another preferred embodiment, methods (also referred to herein as “screening assays”) are provided for identifying modulators, i.e., candidate or test compounds or agents (e.g., proteins, peptides, peptidomimetics, peptoids, small molecules or other drugs) which modulate the interaction of a DEP domain of a member of an R7 family of a regulator of G protein signaling (RGS7) and muscarinic acetylcholine M3 receptor (M3R), etc. Compounds thus identified can be used to modulate the activity of target gene products, prolong the half-life of a protein or peptide, regulate cell division, etc, in a therapeutic protocol, to elaborate the biological function of the target gene product, or to identify compounds that disrupt normal target gene interactions.

An agonist and/or antagonist of Gβ₅ complex activity identified by the methods of the invention may be a small molecule, a chemical, a peptide, a peptidomimetic, a region of the natural ligand for a G protein subunit or an RGS protein, a region of a G protein subunit or an RGS protein, or any other compound that mimics a ligand for a G protein subunit or RGS protein. The agonist may also be a partial agonist that exhibits partial enhancement of Gβ₅ complex activity, and the antagonist may be a partial antagonist that exhibits partial inhibition of Gβ₅ complex activity.

After identifying a test compound or candidate agent as an agonist and/or an antagonist, the compound may then be used to treat subjects with diseases and disorders associated with Gβ₅ complex activity. An identified agonist may be used to treat diseases and disorders in which Gβ₅ complex activity is diminished, for example due to missing, depleted, or defective Gβ₅, RGS7, or R7BP. An identified antagonist may be used to treat diseases and disorders in which Gβ₅ complex activity needs to be inhibited. Due to the importance of their role as regulators of G protein signaling, RGS proteins are therapeutic targets for the treatment of diseases and disorders such as diseases and disorders of neuronal function and of visual function, psychiatric disorders, Parkinson's disease, cardiovascular diseases and disorders, and drug addiction disorders (for a review, see 40).

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the one-bead one-compound library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Nat'l Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In another preferred embodiment, the candidate therapeutic agent comprises, proteins, peptides, organic molecules, inorganic molecules, nucleic acid molecules, and the like. These molecules can be natural, e.g. from plants, fungus, bacteria etc., or can be synthesized or synthetic.

A prototype compound may be believed to have therapeutic activity on the basis of any information available to the artisan. For example, a prototype compound may be believed to have therapeutic activity on the basis of information contained in the Physician's Desk Reference. In addition, by way of non-limiting example, a compound may be believed to have therapeutic activity on the basis of experience of a clinician, structure of the compound, structural activity relationship data, EC₅₀, assay data, IC₅₀ assay data, animal or clinical studies, or any other basis, or combination of such bases.

A therapeutically-active compound is a compound that has therapeutic activity, including for example, the ability of a compound to induce a specified response when administered to a subject or tested in vitro. Therapeutic activity includes treatment of a disease or condition, including both prophylactic and ameliorative treatment. Treatment of a disease or condition can include improvement of a disease or condition by any amount, including prevention, amelioration, and elimination of the disease or condition. Therapeutic activity may be conducted against any disease or condition, including in a preferred embodiment against human immunodeficiency virus, cancer, arthritis or any combination thereof. In order to determine therapeutic activity any method by which therapeutic activity of a compound may be evaluated can be used. For example, both in vivo and in vitro methods can be used, including for example, clinical evaluation, EC₅₀, and IC₅₀ assays, and dose response curves.

Candidate compounds for use with an assay of the present invention or identified by assays of the present invention as useful pharmacological agents can be pharmacological agents already known in the art or variations thereof or can be compounds previously unknown to have any pharmacological activity. The candidate compounds can be naturally occurring or designed in the laboratory. Candidate compounds can comprise a single diastereomer, more than one diastereomer, or a single enantiomer, or more than one enantiomer.

Candidate compounds can be isolated, from microorganisms, animals or plants, for example, and can be produced recombinantly, or synthesized by chemical methods known in the art. If desired, candidate compounds of the present invention can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries. The other four approaches are applicable to polypeptide, non-peptide oligomer, or small molecule libraries of compounds and are preferred approaches in the present invention. See Lam, Anticancer Drug Des. 12: 145-167 (1997).

In an embodiment, the present invention provides a method of identifying a candidate compound as a suitable prodrug. A suitable prodrug includes any prodrug that may be identified by the methods of the present invention. Any method apparent to the artisan may be used to identify a candidate compound as a suitable prodrug.

In another aspect, the present invention provides methods of screening candidate compounds for suitability as therapeutic agents. Screening for suitability of therapeutic agents may include assessment of one, some or many criteria relating to the compound that may affect the ability of the compound as a therapeutic agent. Factors such as, for example, efficacy, safety, efficiency, retention, localization, tissue selectivity, degradation, or intracellular persistence may be considered. In an embodiment, a method of screening candidate compounds for suitability as therapeutic agents is provided, where the method comprises providing a candidate compound identified as a suitable prodrug, determining the therapeutic activity of the candidate compound, and determining the intracellular persistence of the candidate compound. Intracellular persistence can be measured by any technique apparent to the skilled artisan, such as for example by radioactive tracer, heavy isotope labeling, or LCMS.

In screening compounds for suitability as therapeutic agents, intracellular persistence of the candidate compound is evaluated. In a preferred embodiment, the agents are evaluated for their ability to modulate the protein or peptide intracellular persistence may comprise, for example, evaluation of intracellular residence time or half-life in response to a candidate therapeutic agent. In a preferred embodiment, the half-life of a protein or peptide in the presence or absence of the candidate therapeutic compound in human tissue is determined. Half-life may be determined in any tissue. Any technique known to the art worker for determining intracellular persistence may be used in the present invention. By way of non-limiting example, persistence of a compound may be measured by retention of a radiolabeled or dye labeled substance.

A further aspect of the present invention relates to methods of inhibiting the activity of a condition or disease comprising the step of treating a sample or subject believed to have a disease or condition with a prodrug identified by a compound of the invention. Compositions of the invention act as identifiers for prodrugs that have therapeutic activity against a disease or condition. In a preferred aspect, compositions of the invention act as identifiers for drugs that show therapeutic activity against conditions including for example cancer, inflammation, rheumatoid arthritis, autoimmune diseases, neurological diseases, immunosuppression and the like, or any combination thereof. Compositions of the invention may also act as identifiers for drugs that have therapeutic activity against infectious agents. Infectious agents against which the therapeutic agents may be effective include, without limitation, bacteria, viruses, and yeast.

If desired, after application of an identified prodrug, the amount of an infectious organism or the level or any material indicative of the infection or condition may be observed by any method including direct and indirect methods of detecting such level. Quantitative, semi-quantitative, and qualitative methods of determining such a level are all contemplated. Any method, including but not limited to, observation of the physiological properties of a living organism, are also applicable.

In one embodiment, a screening assay is a cell-based assay in which a cell expresses a protein- or peptide-detectable marker construct or fusion protein construct, for example, Gβ5-RGS7 complex, GST-R7DEP, Gβ₅, RGS7, or R7BP fusion molecules (e.g. GST, luciferase fusion partners), or mutants thereof, which is contacted with a test compound, and the ability of the test compound to modulate the modulates the interaction of a DEP domain of a member of an R7 family of a regulator of G protein signaling (RGS7) and muscarinic acetylcholine M3 receptor (M3R) is determined. Determining the ability of the test compound to modulate the interaction of a DEP domain of a member of an R7 family of a regulator of G protein signaling (RGS7) and muscarinic acetylcholine M3 receptor (M3R) can be accomplished by monitoring, for example, Ca²⁺ assays, pull-down assays, etc, assays described in detail in the Examples section which follows. The cell, for example, can be of mammalian origin, e.g., human. Any one or more of the above constructs can be used.

In another preferred embodiment, the screening assay is a high-throughput screening assay.

In another preferred embodiment, soluble and/or membrane-bound forms of isolated proteins, mutants or biologically active portions thereof, can be used in the assays if desired. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON™ X-100, TRITON™ X-114, THESIT™, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl═N,N-dimethyl-3-ammonio-1-propane sulfonate.

Cell-free assays can also be used and involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al, U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorometric detection means well known in the art (e.g., using a fluorimeter).

In another embodiment, determining the ability of a protein to bind or “dock” to a target molecule or docking site on a target molecule can be accomplished using real-time Biomolecular Interaction Analysis (BIA) (see, e.g., Sjolander, S, and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BLAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target product or the test substance is anchored onto a solid phase. The target product/test compound complexes anchored on the solid phase can be detected at the end of the reaction. Preferably, the target product can be anchored onto a solid surface, and the test compound, (which is not anchored), can be labeled, either directly or indirectly, with detectable labels discussed herein.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

### Obesity:

The inventors have surprisingly discovered that age-related obesity is also associated with Gβ₅ complex (Example 2). In particular, it was discovered that lowered expression of Gβ₅ complexes, for example due to the presence of a single copy of a Gβ₅ gene or due to a mutation in a Gβ₅ gene, results in the age-related onset of obesity. Accordingly, another aspect of the invention provides novel compounds and compositions for the treatment of obesity and methods for the identification of these compounds. An example of a method of identifying a compound capable of modulating weight gain provided by the invention comprises administering a test compound to a first mouse comprising a deletion of one allele of a gene encoding a Gβ₅ protein, and comparing weight gain of the first mouse to the weight gain of a second mouse comprising the deletion of one allele of a gene encoding a Gβ₅ protein not administered the test compound, wherein a difference in weight gain between the first mouse and the second mouse identifies that the test compound is capable of modulating weight gain. The first mouse and second mouse may be Gβ₅ heterozygous mice or RGS7 heterozygous mice. Yet another aspect of the invention provides novel animal models for studies of obesity: animals with lower expression or impaired activity of Gβ₅-RGS complex for studies of associated physiologic processes, effects of drug treatments, and diet and exercise regiments.

The invention also provides methods for predicting the onset of obesity in an individual. Lowered expression of Gβ₅ complex proteins, such as Gβ₅, RGS7, or R7BP results in the reduction of the amount of Gβ₅ complex. Therefore, genetic mutations leading to a lower expression of any of the encoded proteins may result in age-related obesity. In support of this notion, a recent study showed a potential link between some types of obesity and the RGS7 gene in humans (41). Accordingly, an aspect of the invention provides methods for identifying mutations in Gβ₅ complex genes and correlating an identified mutation with the prediction of the onset of obesity in an individual carrying such a mutation.

Without wishing to be bound by theory, recent studies by the Inventors provided new ideas that potentially explain the role of Gβ5 with the Gβ5-R7 dimer. First, they found that Gβ5 binds to the DEP domain within the dimer; as described below in more detail, the affinity of this interaction is low compared to the interaction of Gb5 with the GGL domain, which might underlie dynamic changes in the conformation within this molecule (21). Second, the Inventors identified a novel mechanism how Gb5-RGS7 can modulate the activity of some G protein-coupled receptors (Example 3). This study showed that Gβ5-RGS7 complex can directly bind to the receptor. This interaction is highly selective and so far was found to occur only with muscarinic M3 receptor, but not with the muscarinic M1 receptor. The interaction occurs between the third intracellular loop of M3 receptor and the DEP domain of RGS7. This novel mechanism of inhibition of the Gq-mediated signal from the M3 receptor does not involve the GAP activity of RGS7.

### Administration of Compositions to Patients

The compositions or agents identified by the methods described herein may be administered to animals including human beings in any suitable formulation. For example, the compositions for modulating protein degradation may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

The compounds can be administered with one or more therapies. For example, chemotherapy, chemokines, radionuclides, cytokines, anti-angiogenic agents or radiotherapy. The compositions provided herein may be used alone or in combination with conventional therapeutic regimens such as surgery, irradiation, chemotherapy and/or bone marrow transplantation (autologous, syngeneic, allogeneic or unrelated). The chemotherapeutic agents may be administered under a metronomic regimen. As used herein, “metronomic” therapy refers to the administration of continuous low-doses of a therapeutic agent. Therapeutic agents can include, for example, chemotherapeutic agents such as, cyclophosphamide (CTX, 25 mg/kg/day,p.o.), taxanes (paclitaxel or docetaxel), busulfan, cisplatin, cyclophosphamide, methotrexate, daunorubicin, doxorubicin, melphalan, cladribine, vincristine, vinblastine, and chlorambucil.

In another preferred embodiment, one or more Gβ5-RGS7 complex molecules can be linked or fused with one or more agents such as growth factors, protein inhibitors, cytokines and the like.

Dosage, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

### Formulations

While it is possible for a composition to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0.1% to 1% w/w of the formulation. The topical formulations of the present invention, comprise an active ingredient together with one or more acceptable carrier(s) therefor and optionally any other therapeutic ingredients(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

Reference will now be made to specific examples illustrating the constructs and methods above. It is to be understood that the examples are provided to illustrate preferred embodiments and that no limitation to the scope of the invention is intended thereby.

### EXAMPLES

### Example 1

### Intra-Molecular Interaction Between the DEP Domain of RGS7 and the Gβ₅ Subunit

A. Recombinant DEP Domain of RGS7 Binds to Native Gβ₅ and Gβ₁ Complexes

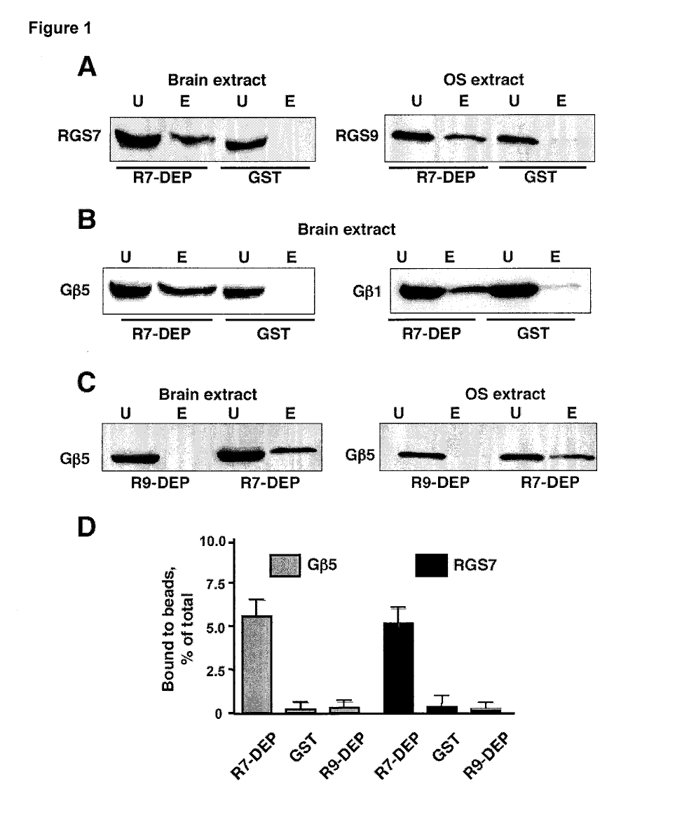

To identify the potential binding partners of the DEP domain of RGS7, a GST fusion construct containing the amino acids 34-124 of bovine RGS7, termed GST-R7DEP or R7-DEP, was generated. Using Glutathione-Sepharose coupled to this GST fusion protein as an affinity matrix for the analysis of brain extracts, it was unexpectedly found that it retained native RGS7-Gβ₅ complexes (FIG. 1A, B). Similarly, R7-DEP bound to the Gβ_{5L}-RGS9 complex solubilized from the preparations of bovine photoreceptor outer segments (OS). The Gβ₅-RGS complexes from either source did not bind to the beads with GST or the GST fusion of the putative DEP domain of RGS9 (amino acids 20-117), indicating that the interaction of Gβ₅-RGS complexes with R7-DEP was specific. The eluates from R7-DEP beads did not contain any distinct proteins detectable by coomassie or silver stain. However, the antibody against Gβ₁ revealed its presence in the eluates from the R7-DEP beads (FIG. 1B), suggesting that the DEP domain cannot distinguish between the Gβ subunit subtypes.

B. R7-DEP Interacts with Recombinant Gβ₅ and Gβ₁

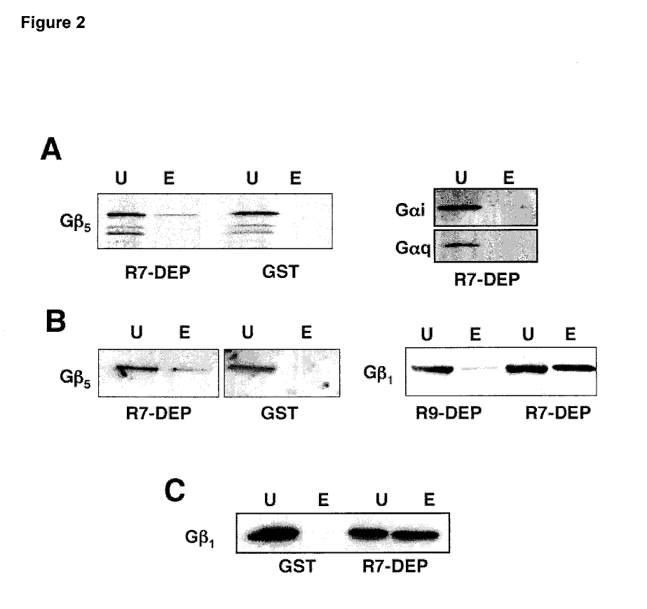

To determine which entities—Gβ, Gγ, or RGS—are responsible for the interaction with the RGS7 DEP domain, GST-R7DEP was used for the pull-down of recombinant Gβ₅ and Gβ₁ subunit complexes. FIG. 2A shows that in vitro translated Gβ₅ specifically bound to the beads with GST-R7DEP, but not to the beads with GST. In contrast, other in vitro translated proteins, such as Gα subunits, Gαᵢ or Gα_{q}, did not bind to R7-DEP under the same conditions. Similar to in vitro translated Gβ₅, Gβ subunits type 1 or 5 transiently expressed in cultured COS-7 or HEK 293 cells also associated with the DEP domain of RGS7. Like their native counterparts (FIG. 1), these recombinant Gβ subunits did not bind to GST or the DEP domain of RGS9, supporting the specificity of the interaction with RGS7 DEP. Importantly, the interaction of either Gβ₁ or Gβ₅ with R7-DEP was unaffected by the presence of Gγ. Although it cannot be ruled out that (a fraction of) the Gβ subunits was associated with endogenous Gγ subunits present in either the transfected cells or reticulocyte lysate, this observation indicated that the Gβ is sufficient for interaction with the DEP domain. To rule out the potential contribution of any additional molecules present in the cellular extracts or reticulocyte lysate, a purified recombinant Gβ_{1γ2} complex (FIG. 2C) was used. A robust interaction of this Gβγ complex with the RGS7 DEP domain was detected. Taken together, these results indicate that the DEP domain of RGS7 can directly bind to G protein β subunits, apparently regardless of their subtype.

C. The Interaction Between RGS7 DEP and Gβ₅ is Dynamic

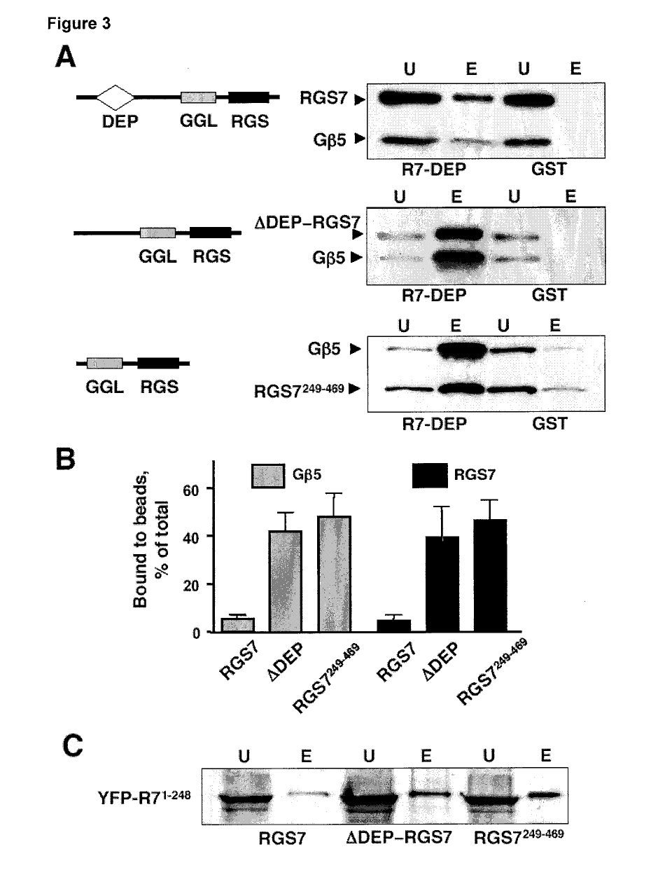

The data on brain and OS extracts suggested that recombinant R7-DEP binds to the native Gβ₅-RGS complexes, which implied that the presence of the endogenous DEP domain could not completely prevent this interaction. One of the mechanisms allowing this interaction could be the competition of the exogenous R7-DEP with the endogenous DEP domain for the same site on Gβ₅. In this scenario, the deletion of the intrinsic DEP domain should enhance the association of R7-DEP with a DEP-less Gβ₅-RGS7 complex. To test this, two RGS7 constructs lacking the DEP domain were prepared: ΔDEP-RGS7, which lacks amino acids 34-125 (putative DEP domain), and RGS7²⁴⁹⁻⁴⁶⁹, which lacks the first 248 amino acids encompassing the DEP domain along with the N-terminus and the linker region (FIG. 3). These constructs were transiently expressed in COS-7 cells together with Gβ₅ and the resulting heterodimers were subjected to the GST pull-down assay using R7-DEP. The data showed that both DEP-less constructs bound to GST-R7DEP much more efficiently than the full-length RGS7-Gβ₅ (FIG. 3A). The increased efficiency of the R7-DEP pull-down may be a result from the lack of competition from the intrinsic DEP domain.

To confirm this conclusion, a reciprocal pull-down was used, where the DEP domain was soluble and the Gβ₅-RGS complexes were immobilized via an antibody against the C-terminus of RGS7 (FIG. 3C). In this alternative assay, the N-terminal portion of RGS7 encompassing the DEP domain and the linker region (amino acids 1-248) fused to the C-terminus of yellow fluorescent protein, YFP-R7¹⁻²⁴⁸, was utilized. YFP-R7¹⁻²⁴⁸ was co-transfected in COS-7 cells together with Gβ₅ and either full-length RGS7, ΔDEP or RGS7²⁴⁹⁻⁴⁶⁹. The cell lysates were immunoprecipitated with the antibody against the C-terminus of RGS7 and the fractions were probed for the presence of YFP-R7¹⁻²⁴⁸, YFP-R7¹⁻²⁴⁸ was retained by the Protein-A beads with anti-RGS7 antibody, showing that the N-terminal portion of RGS7, apparently via the DEP domain, can physically associate with the Gβ₅-RGS7 dimers. Furthermore, co-immunoprecipitation tended to be more efficient with the DEP-less RGS7 constructs, supporting the data from the GST pull-down experiments (FIG. 3A). Together, these results indicate that the exogenously added DEP domain competes with the intrinsic DEP domain present in full length RGS7, for binding to Gβ₅.

To permit the association of exogenously added DEP domain with Gβ₅, the intra-molecular interaction between the intrinsic DEP and Gβ₅ must be dynamic and have relatively low affinity. To investigate this hypothesis, a protein-protein binding assay based on fluorescence resonance energy transfer (FRET) was devised. The advantage of this approach is that the interaction is detected in solution, and does not require trapping of the protein complex on a solid phase, which may be inefficient for low affinity complexes. Resonance energy transfer occurs when the emission spectrum of a fluorophore (donor) overlaps with the absorption spectrum of an acceptor molecule. In FRET, the energy from the photo-excited fluorophore (“donor”) is not emitted, but is transferred to the acceptor fluorophore, which then emits at lower energies. This energy transfer occurs if the distance between the pair of fluorophores is less than 100 angstroms. The efficiency of FRET depends on the extent of spectral overlap, the orientation of the fluorophores, and drops exponentially as the distance between the fluorophores increases. Therefore, FRET may be used to study the physical interaction between a pair of fluorescently tagged molecules. The previously characterized CFP-Gβ₅ fusion was employed as the energy donor (28); the YFP-RGS7 was used as the FRET energy acceptor. No FRET was registered between CFP-Gβ₅ and YFP or YFP-RGS7 constructs and CFP.

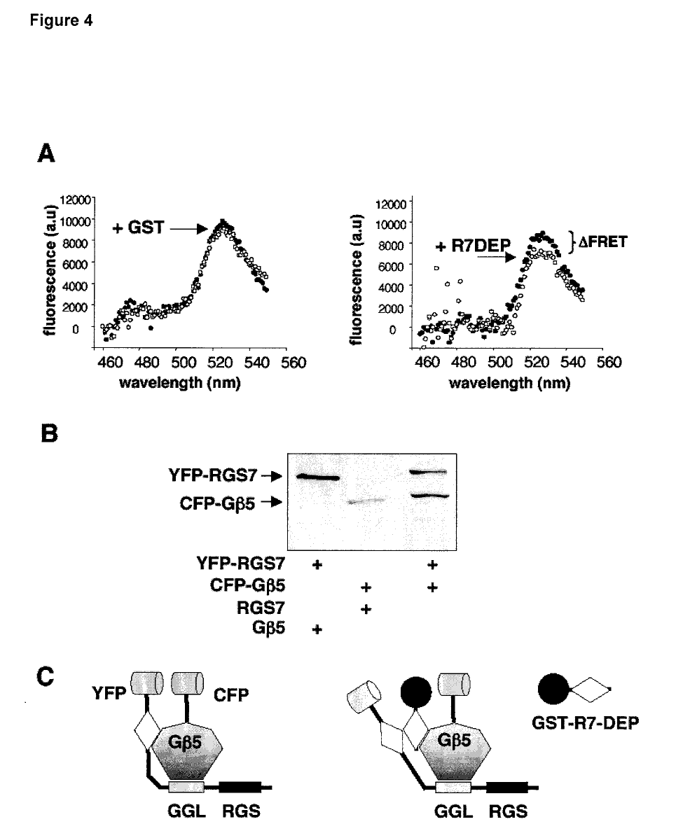

GST-R7DEP, but not GST alone, decreased FRET (FIG. 4A), pointing to the dynamic nature of the DEP-Gβ₅ association within the Gβ₅-RGS dimer. The FRET reduction did not exceed 15-20% of the total FRET signal.

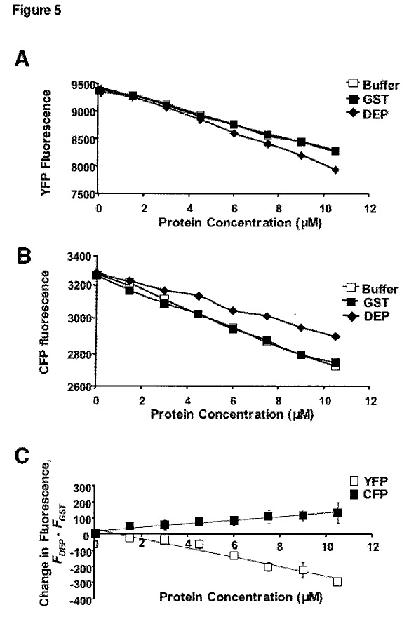

Measuring FRET requires subtraction of CFP fluorescence in the YFP emission channel “bleed-through” and of YFP background fluorescence from the spectra obtained from cells expressing both CFP-Gβ₅ and YFP-RGS7. Therefore, additional spectra must be recorded from control cells expressing only CFP-tagged and only YFP-tagged molecules. The inherent variability of protein expression in transient transfection, and, in particular, the presence of fluorescent protein degradation products in the crude lysates, made the calculations of FRET and comparison of different experiments difficult. The present inventors performed a series of experiments using the same lysate of COS-7 cells expressing full-length YFP-RGS7 and CFP-Gβ₅. The lysate was split in three aliquots (FIG. 5). To one aliquot, the increasing concentrations of recombinant R7-DEP were added. To second aliquot, the increasing concentrations of GST were added as a control. To third aliquot, buffer in which these proteins were prepared was added for the control of the dilution of the fluorescent lysate. Each lysate was excited at CFP excitation maximum (433 nm) and the change in total YFP fluorescence (525 nm) was measured upon the sequential addition of recombinant R7-DEP (FIG. 5). The advantage of this simplified approach is that the subtraction of the YFP background fluorescence and CFP bleed-through was not necessary. To account for the effect of the sample dilution, which occurs when the GST-R7DEP or GST is added to the cell lysate, an aliquot of the same lysate was titrated with the corresponding buffer. Addition of R7-DEP resulted in a small reduction of total YFP fluorescence level (FIG. 5). This reduction occurred very quickly, supporting the notion that the association between DEP and Gβ₅ is a dynamic equilibrium, with a fairly rapid off-rate.

D. The Putative Binding Site for Gβ₅ on RGS7 DEP Domain

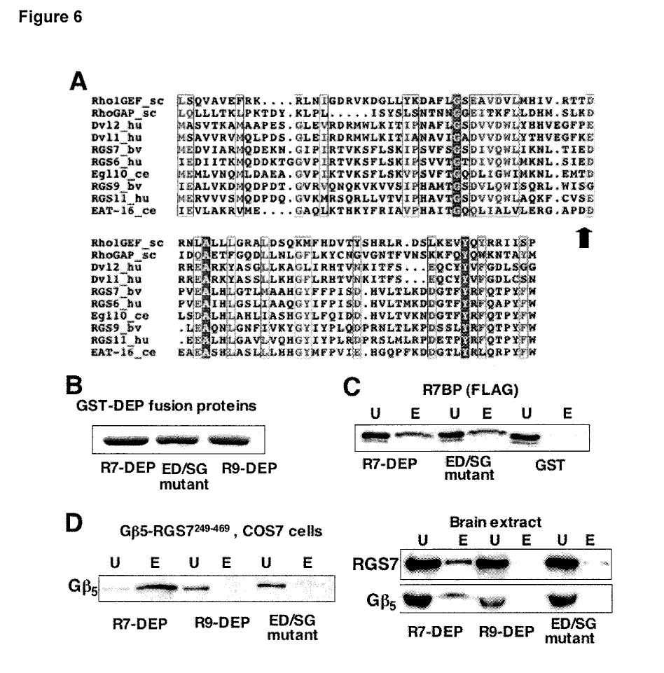

An important step in the characterization of a protein-protein interaction is the identification of the binding site through generation of interaction-deficient mutants. The design of an R7-DEP mutant incapable of Gβ₅ binding was aided by the finding that the DEP domain of RGS9 does not bind to Gβ₅. The alignment of DEP domains revealed that the most striking difference in the distribution of charged amino acids was the absence in RGS9 of the negative charge at the position corresponding to Asp-74 in RGS7 (FIG. 6A). A negatively charged amino acid is present at this position in all R7RGSs except RGS9, and even in the DEP domains of Dishevelled, Rho1GEF and RhoGAP. It was hypothesized that the negative charge is important for the interaction with Gβ₅, and replaced Glu73 and Asp74 of RGS7, with Ser and Gly residues, which are present at the corresponding positions in the DEP domain of RGS9. This ED/SG mutant was expressed as a GST fusion in E. coli and utilized in pull-down assays using extracts of mouse brain and COS-7 cells expressing Gβ₅ associated with RGS7 and RGS7²⁴⁹⁻⁴⁶⁹ (FIG. 6B). These experiments showed that there was at least a 10-fold reduction in the amount of Gβ₅ that was pulled down by the mutant relative to the wild-type DEP domain of RGS7. The ED/SG mutant, however, retained the ability to bind R7BP (FIG. 6C), which showed that the overall folding of the DEP domain was not compromised by the mutation, and that R7BP and the Gβ subunits likely associate with two distinct surfaces of the DEP domain.

E. The Effect of R7BP the E73S/D74G Double Mutation (ED/SG) on the Function of the Gβ₅-RGS7 Complex

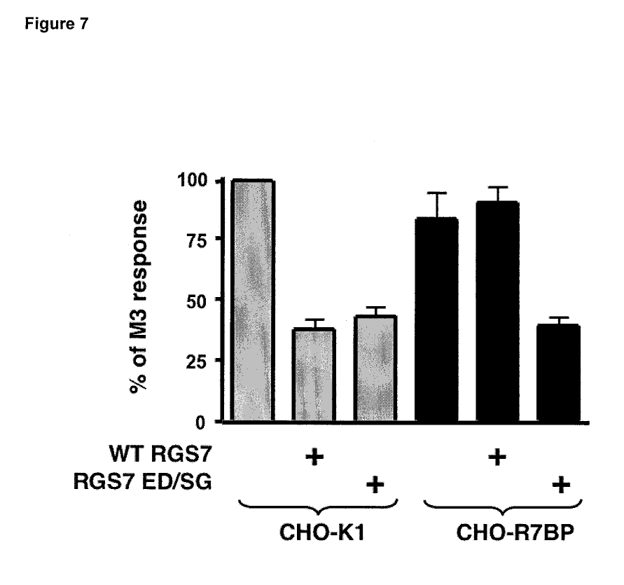

Previous studies showed that the Gβ₅-RGS7 complex can attenuate Gq-mediated Ca²⁺ response to muscarinic M3 receptor stimulation in transfected cells (8, 28). Here, this assay was used to study the effect of the ED/SG mutation on the function of full-length RGS7 protein (FIG. 7). The RGS7^{ED/SG} mutant was transiently expressed together with Gβ₅ and the M3 receptor in CHO-K1 cells. To test the role of R7BP, the CHO cell line stably expressing R7BP, CHO-R7BP was used. The rationale behind using the stable R7BP transfection was to limit the number of transiently transfected cDNAs to three. Western blot analysis showed that similar to the wild-type RGS7, RGS7^{ED/SG} mutant was localized to the membranes in the presence of R7BP, showing that the ED/SG mutation did not affect the R7BP interaction with the Gβ₅-RGS7 dimer. The changes in free Ca²⁺ concentration in response to Carbachol using the ratiometric dye, fura-2 in control CHO-K1 and in the CHO-R7BP cells were recorded. The results of these recordings (FIG. 7) revealed two surprising and unexpected novel effects. First, it was found that R7BP prevented Gβ5-RGS7 from exerting its negative effect on the Carbachol-induced calcium mobilization. This negative effect of R7BP on Gβ5-RGS7 in this Gq-mediated pathway contrasts with its positive effect on Gβ5-RGS7 reconstituted with Gi and GIRK channel in Xenopus oocytes (29). Second, the experiments highlighted the potential function of the “open” conformation. In the absence of R7BP, the mutant and wild-type RGS7 complexes reduced the amplitude Ca²⁺ response to the same degree (FIG. 7, grey bars). However, in the presence of R7BP, a dramatic difference was observed (FIG. 7, black bars). In contrast to the wild-type Gβ₅-RGS7-R7BP trimer, the RGS7^{ED/SG} mutant reduced the amplitude of Ca²⁺ transients by more than 50%. These results indicate that the inhibition of signal transduction by Gβ₅-RGS7 occurs primarily when the dimer is in its open conformation. R7BP, which facilitates DEP-Gβ₅ binding and the “closure” of the dimer, prevents Gβ₅-RGS7 from inhibiting this M3-mediated Ca²⁺ mobilization pathway.

F. Mutations in Both Gβ₅ and RGS7 DEP Domain can Prevent Binding Between these Two Proteins

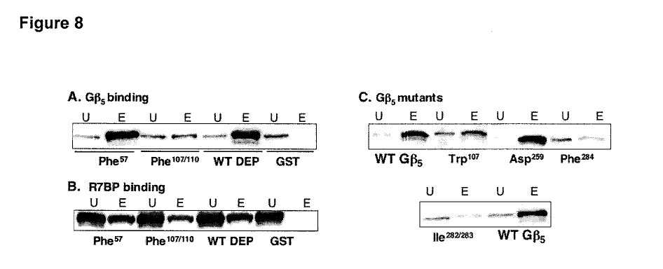

Several other mutations were tested for the ability to prevent the protein-protein interaction between Gβ₅ and the DEP domain of RGS7. The results (FIG. 8) show that mutations in both RGS7 (i.e., double-mutant Phe107/Phe110 substitution for two Ala), and Gβ₅ (i.e., Trp107 or the double mutant Ile282/Ile283 for Ala), can prevent the interaction. At the same time, Phe107/Phe110 mutations have no effect on binding to R7BP, indicating that the overall folding of the DEP domain is intact (FIG. 8B).

G. The Protein-Protein Interaction Between Gβ5 and the DEP Domain of RGS7 can be Blocked by a Small Chemical.

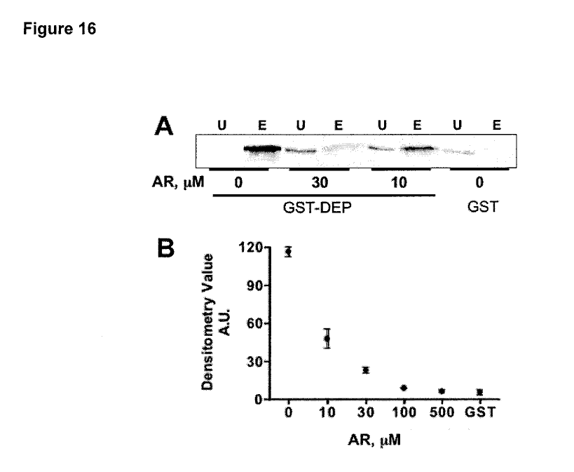

A small set of small chemicals of organic dye family were tested as potential inhibitors of the DEP:Gb5 interaction. The compounds were introduced to the pull-down assay where the GST-DEP fusion protein was immobilized on the glutathione beads and Gβ5-R²⁴⁹ was present in the soluble phase. Some of the tested compounds, such as suramin, are known to be disruptors of many protein-protein interaction and some resembled the chemicals influencing Gβ subunit function (30). Three structurally related compounds blocked the DEP-Gβ5 pull-down; one of these compounds, code-named “AR”, had IC₅₀ of ˜10 μM in the pull-down assay (FIG. 16).

H. Discussion

Specificity of DEP domain-Gβ interaction. The interaction of the DEP domain of RGS7 with Gβ subunits was discovered through the utilization of a GST pull-down assay (FIGS. 1, 2 and 8). The results of this experiment met the basic criteria of binding specificity, such as the lack of Gβ retention on the beads with immobilized GST, and the lack of association of GST-R7DEP fusion with the bulk of the proteins in the cellular lysates and with key molecules such as Gα subunits. The specificity of the GST-R7DEP pull-down with Gβ₅ was corroborated by alternative assays such as immunoprecipitation and FRET (FIGS. 3-5), as well as mutagenesis (FIG. 8).

Despite the reasonably high homology between the DEP domains of RGS7 and RGS9, the GST fusion of the RGS9 DEP domain did not bind to Gβ subunits under the same conditions (FIG. 1C). This finding supported the specificity of the R7-DEP interaction with Gβ subunits even further, and provided a strategy to determine the structural basis of the interaction. The NMR structure of the DEP domain of mDv11 revealed an electric dipole, consisting of three charged residues (K434, D445, D448), on an exposed surface of this protein, and it was previously suggested that this dipole was important for interactions with other proteins (31). One possibility is that a similar dipole surface is also presented by the surface of the RGS7 DEP domain. The focus on a particular conserved aspartic acid, D74 in RGS7, was due to its presence in all R7RGS proteins and even in other proteins such as RhoGAP and Dishevelled, but is replaced with a serine residue in RGS9. In RGS7, as well as RGS6, Egl-10 and Eat 16, this conserved Asp is adjacent to a Glu residue, presumably creating a significant negative charge. The substitution of these two amino acids, Glu and Asp with the corresponding amino acids in RGS9 (Ser and Gly), was sufficient to diminish the DEP-Gβ₅ interaction, strongly indicating that the negative charge is essential for binding Gβ₅ (FIG. 6B). Importantly, this mutation did not affect binding of the DEP domain to R7BP, indicating that the overall structure of the molecule remained intact. Considering the location of E73/D74 of RGS7 in the putative loop region between helices 2 and 3 and the currently available solution structure data for a similar DEP domain, these residues are predicted to constitute a bona fide binding site for Gβ₅.

Since the monomeric Gβ₅ subunit can bind to GST-R7DEP (FIG. 2), the binding site for DEP domain is likely to be present on the Gβ chain rather than within the GGL domain. Since Gβ₁ and Gβ₅ subunits were similar in their binding to the RGS7 DEP domain it is likely that the binding site is located in a conserved region of Gβ. This study focuses on the hypothesis that this Gβ-R7DEP interaction represents the intra-molecular association between the Gβ₅ subunit and the DEP domain. Indeed, it appears that unless an additional mechanism can reduce the DEP-Gβ₅ binding affinity, the interaction between the intrinsic entities would be favored over the interaction with a competing free Gβγ. However, at this point it cannot be ruled out that the DEP domain of RGS7 or other DEP domains can interact with canonical Gβγ subunit complexes originating from the activated G proteins.

“Open” and “closed” conformations of the Gβ₅-RGS7 complex. The pull-down of the full-length RGS7-Gβ₅ complex by R7-DEP (FIGS. 1, 3), the enhanced binding of the DEP-less constructs (FIG. 3), and FRET assays (FIGS. 4, 5), all strongly indicate that the endogenous DEP domain can be displaced from Gβ₅/GGL, and that this interaction is dynamic in nature. It was found that the Gβ₅-RGS7 complex can exist in (at least) two distinct conformations: “open”, when the DEP domain does not make contact with Gβ₅ and “closed”, when the DEP domain is in physical contact with Gβ₅.

Importantly, the fact that native Gβ₅-RGS7 and Gβ₅-RGS9 complexes do interact with R7-DEP indicates that a portion of the native complex exists in the open conformation. According to the estimates based on the quantification of the pull-down data, this fraction does not exceed 5-10% of the total Gβ₅-RGS7 amount (FIG. 1D). However, because this pool appears to represent the “active” state of RGS7 complex, it is likely to be significant. The open conformation may be induced within the R7BP-RGS7-Gβ₅ heterotrimer by a post-translational modification or by interaction with a protein or a specific lipid. Alternatively, the open state is simply the Gβ₅-RGS7 dimer, since its behavior resembles the behavior of Gβ₅-RGS7 expressed in vitro. Indeed, Hepler and colleagues demonstrated that RGS7 can associate with insect cell membranes through direct palmitoylation of RGS7 (32).

Role of the Gβ₅-DEP interaction. The Gβ₅-R7 complexes were discovered in 1998 (7, 13), but understanding the true significance of the presence of Gβ₅ within the RGS molecule proved to be difficult (12, 17). The role of Gβ₅ subunit in the Gβ₅-RGS complex was addressed by comparing the function of monomeric recombinant RGS7 with Gβ₅-RGS7. Reconstitution in transfected cells or Xenopus oocytes was difficult to interpret because of the profound effect of Gβ₅ on the expression level of the RGS subunit (8, 28, 33, 34). Once in vitro studies with recombinant proteins circumvented this problem, it was shown that Gβ₅ reduced the interaction of RGS7 with Gαₒ (14), and accordingly, the GAP activity of RGS9 toward Gαt was reduced by Gβ₅ (35, 36). It was also found that other domains of RGS9, including the DEP domain-containing N-terminus, can counteract the Gβ₅-mediated inhibition, and make the GAP activity of the complex more selective to the Gα-GTP-effector complex compared to just Gα-GTP (35, 36).

To explore the significance of the DEP-Gβ₅ interaction the putative Gβ₅ binding site on RGS7 DEP domain was mutated and the functional activities of the resulting Gβ₅-RGS7 and Gβ₅-RGS7-R7BP complexes were tested (FIG. 7). These results showed that like wild-type RGS7, RGS7^{ED/SG} mutant attenuated M3 receptor-induced Ca²⁺ mobilization. However, the mutant retained its activity in the presence of R7BP, whereas R7BP abolished the activity of the wild-type RGS7. The inference is that when Gβ₅ and DEP are dissociated and the complex is in its open conformation, it is active with respect to inhibition of Gq-mediated signaling. When Gβ₅ and DEP domains interact, the molecule assumes the closed conformation, which is inactive. The idea of allosteric control of the RGS7 complex's GAP activity by its non-RGS domains is consistent with what is thought about the regulation of RGS9 GAP activity toward transducin (36, 37).

In addition to the regulation of RGS7-mediated effects on G protein signaling, the DEP-Gβ₅ interaction may play other role(s). For example, it is possible that the DEP-Gβ₅ interaction plays a role in the nuclear localization of Gβ₅-RGS7 (29, 38, 39) or the potential relationship between Gβ₅-R7 and SNARE complex (40). Since Gβ₅ localizes to the nucleus when reconstituted with RGS7 but not Gγ (39), it is possible that the Gβ₅-DEP, rather than the Gβ₅-GGL, interaction is essential for nuclear targeting. The recently discovered interaction of Gβγ with SNARE complexes (41, 42) hints at the influence that Gβ₅ may have on the interaction of DEP with snapin or R7BP, which itself slightly resembles SNARE proteins (29). Such an effect of Gβ₅ could play a role in neuronal vesicular trafficking and fusion.

H. Materials and Methods

Antibodies. Affinity-purified anti-peptide rabbit polyclonal antibodies raised against RGS7, RGS9, Gβ₅, and Gβ₁ have been described earlier (8). Antibody against GFP was from Clontech and anti-FLAG antibody was from Sigma.

Cloning of GST-DEP constructs. The following constructs were generated for bacterial expression and subsequent purification.

GST R7-DEP: Nucleotides 100-372 (corresponding to amino acids 34-124 of bovine RGS7) were PCR amplified from full-length RGS7 using the forward primer 5′-GGAATTCATGCAAGATGAAAAAAACGGA-3′ (SEQ ID NO:1) and the reverse primer 5′-GGAAGCTTTCAGTGATGGTGATGGTGATGTTCCGGCTCCCAACAATTT-3′ (SEQ ID NO:2). This fragment was cloned into pGEX-KG vector linearized with EcoRI/HindIII. The double mutation, E73S/D74G, was introduced using the forward primer 5′-AAGAACTTAACCATAAGCGGACCAGTGGAGGCACTC-3′ (SEQ ID NO:3) and the reverse primer 5′-GAGTGCCTCCACTGGTCCGCTTATGGTTAAGTTCTT-3′ (SEQ ID NO:4).

GST R9-DEP: Nucleotides 163 to 456 (corresponding to amino acids 20-117 of bovine RGS9) were amplified using the forward primer 5′-GTGGAATTCTAATCGAGGCCCTTGTGAAGGAC-3′ (SEQ ID NO:5) and the reverse primer 5′-CACGTCGACTTCAGCCGGCCACTGCTGGG-3′ (SEQ ID NO:6). The purified DNA fragment was cloned into pGEX-KG vector at EcoRI and SalI sites.

Purification of GST DEP constructs. One liter bacterial cultures were grown to an OD₆₀₀ of 1.0 at 37° C. Protein expression was induced with the addition of 0.4 mM IPTG for 1.5 to 2 hours at 30° C. Cells were pelleted, and stored at −70° C. until further use. Pellets were resuspended in STE (100 mM Tris pH 8.0, 150 mM NaCl, 1 mM EDTA) buffer containing DNAse, Lysozyme, 5 mM DTT and protease inhibitors. The cell suspension was briefly sonicated on ice. Sarkosyl (final concentration of 1.5%) and Triton X-100 (final concentration of 2%) were added to the cell lysate accompanied by shaking at room temperature for 1 hour. Thereafter the lysate was centrifuged at 19,000 rpm at 4° C. for 30 minutes. The clarified lysate was batch processed using GST-Sepharose 4B beads (GE) using a standard batch-wise procedure described below. Purified protein was desalted and stored at −70° C. with glycerol.

Constructs for Expression in Mammalian Cells.

ΔDEP-RGS7: RGS7 cDNA was cloned into pcDNA3 vector at BamHI and NotI sites. A construct lacking the DEP domain, ΔDEP-RGS7, was generated using PCR mutagenesis for the removal of nucleotides 100-375 corresponding to amino acids 34-125.

RGS7²⁴⁹⁻⁴⁶⁹: This RGS7 construct, which lacks the DEP domain and the linker region, was generated by PCR amplification of nucleotides 745-1410 (corresponding to amino acids 249-469) using the forward primer 5′-CCGGATCCACCATGGAAACTAAACCTCCCACA-3′(SEQ ID NO:7) and the reverse primer 5′-CCGCGGCCGCTTAATAAGACTGAACGAGGCT-3′ (SEQ ID NO:8). The fragment was cloned into pcDNA3 vector linearized with BamHI/NotI.

YFP-R7¹⁻²⁴⁸. Nucleotides 1-744 (corresponding to amino acids 1-248 of the full length bovine RGS7) were amplified using the forward primer 5′-CCAAGCTTATGCAAGATGAAAAAAACGGA-3′ (SEQ ID NO:9) and the reverse primer 5′-CTGAAGCTTTGGTGTGGGTGTGTGGGTAG-3′ (SEQ ID NO:10). The fragment was cloned into pEYFP-N1 vector at HindIII and SalI sites.

Full-length RGS7 ED/SG mutant (RGS7^{ED/SG}). RGS7 cDNA was cloned into pcDNA3 vector at BamHI and NotI sites. The double mutation E73S/D74G was introduced using the same primers used for the GST-R7-DEP double mutation.

Cell culture and transfection. COS-7 cells were cultured in Dulbecco's minimum essential medium supplemented with 10% fetal bovine serum and penicillin/streptomycin. CHO-K1 cells were cultured in F-12K Nutrient Mixture (Kaighn's modification, Gibco) with 10% fetal bovine serum and penicillin/streptomycin. 24 hours prior to transfection, the cells were plated to achieve a density of 0.8×10⁶-1.0×10⁶ cells per 100 mm plate. Transfection was carried out using Lipofectamine 2000 (Invitrogen) as per manufacturer's instructions. The DNA ratio of RGS7 to Gβ5 was maintained at 5:1, with a total of 8.0 μg of DNA per plate. LacZ DNA was used as a control to ensure that the total DNA per plate used in the COS-7 co-transfection assays remained constant. 48 hours after transfection, cells were washed with HBSS, harvested and used for immunoprecipitation, pull-down assays or were pelleted and stored at −70° C. for use in FRET assays.

In vitro translation. To obtain ³⁵S-Met-labeled proteins, rabbit reticulocyte in vitro translation/transcription system (Promega) was used according to the manufacturer's instructions (14).

Preparation of brain homogenates—Mouse brains were homogenized in a lysis buffer (20 mM Tris-HCl pH 7.5, 1 mM EDTA, 50 mM NaCl, 2 mM β-mercaptoethanol) and centrifuged at 50,000×g for 20 min at 4° C. The pellet containing the membranes was washed and resuspended in the same buffer containing 1% sodium cholate. This suspension was left on ice for 30 min, then centrifuged at 50,000×g for 30 min, and the supernatant was used as the membrane extract.

GST pull-down. Glutathione Sepharose 4B beads were pre-washed with PBS+0.1% CHAPS and incubated at 4° C. with purified recombinant GST or the GST fusion proteins for 1 hour, and washed three times with PBS+0.1% CHAPS to remove excess protein. The slurry was incubated for 1-2 hours at 4° C. on a rotary shaker with the various lysates as determined by the experiment. At the end of the incubation, the beads were settled by gravity and the supernatant was collected as the unbound fraction. The resin was extensively washed and then eluted with the addition of SDS-containing sample loading buffer. In a typical assay, the packed volume of the GST resin was 30 μl, and the volume of the protein lysate was 300 μl. The beads were washed three times with 600 μl of PBS+0.1% CHAPS buffer, and eluted with 30 μl of 2×SDS sample loading buffer. The unbound and eluted fractions were resolved by gel electrophoresis and analyzed by western blotting.

Immunoprecipitation from COS-7 cell lysates—48 hours after transient transfection with the required constructs, COS-7 cells were washed with HBSS, and then harvested in lysis buffer (20 mM Tris pH 8.0, 100 mM NaCl, 2 mM MgSO₄, 0.05% Genapol, 5% glycerol and protease inhibitors). The suspension was freeze-thawed, passed through a 19 gauge needle and incubated, with shaking, at 4° C. for 1 hour, and the resulting lysate was centrifuged at 14,000 rpm for 30 minutes. The supernatant was incubated with Protein A Sepharose that had been previously washed and bound to RGS7 antibody (8). After 2-3 hours of incubation on a rotating platform at 4° C., beads were washed and eluted with 2×SDS sample loading buffer. The unbound and immunoprecipitated fractions were analyzed by western blotting.

FRET assay—FRET assays were performed with transiently transfected COS-7 cell lysates (28). COS-7 cells were grown to 70% confluency and transfected in 100 mm plates. Cells were harvested and the lysates were obtained using the same procedures as for immunoprecipitation and GST pull-downs. Protein assays were performed to determine the total protein concentrations in the supernatants, which were then adjusted with PBS to attain the same concentration (typically, 2 mg/ml).

FRET between CFP— and YFP-tagged proteins was determined on a photon counting spectrofluorometer (PTI, Inc.) (28). Briefly, the cell lysates were placed in a 4 ml quartz cuvette, and the spectra were recorded at room temperature with continuous mixing of the lysate with a magnetic stirrer. CFP was excited at 433 nm (2-4 nm slit width, depending on the intensity of fluorescence signal) and the emission spectra were obtained between 465 and 555 nm. The emission peak of YFP was observed at 524-525 nm. For each recording, three spectral scans were performed to obtain the average, which was used in subsequent calculations. The following spectra were recorded. First, the spectra from the lysate containing both the CFP and YFP fusion proteins, i.e., CFP-Gβ5 and YFP-RGS7, were measured. Second, fluorescence was measured from the lysate expressing CFP-Gβ5/RGS7; this control provided us with the measure of CFP fluorescence bleed-through into the YFP emission channels. Third, fluorescence was measured from lysate expressing YFP-RGS7 alone; this was an estimation of the background excitation of YFP at 433 nm. The baseline fluorescence of the cell lysate was determined using cells containing no fluorescently tagged proteins. This “empty” spectrum was subtracted from the spectra recorded from the lysates containing the fluorescent proteins. Then, the “CFP-only” and “YFP-only” spectra were subtracted from the “CFP+YFP” spectrum to detect the increase of YFP fluorescence that occurred due to FRET. These differential spectra are presented in FIG. 4A. In order to correct for the difference between the YFP level in different lysates containing the fusions, for example, CFP-Gβ₅/YFP-RGS7 and untagged Gβ₅/YFP-RGS7, the amount of YFP was determined by measuring the YFP emission when it was excited at 465 nm at which there is maximal YFP excitation. If there was a difference, it was factored into the calculation of the difference between the spectra.

Applying a different method, the change in fluorescence of CFP and YFP was monitored using only one cell lysate containing both the donor and acceptor, CFP-Gβ₅/YFP-RGS7. In this assay, the contribution of CFP bleed-through and background fluorescence was not determined, and therefore the actual FRET value was not calculated. Rather, the specific GST-R7DEP-induced change in total fluorescence was determined. The CFP-Gβ₅/YFP-RGS7 cell lysate was obtained from transfected COS-7 cells and split in three 2 ml aliquots. GST-R7DEP or GST stocks (65 μM), or the storage buffer, was added, with constant stirring, to the cuvette with the cell lysate. The lysate was excited at 433 nm and the emission scanned between 450 and 550 nm using JASCO FP-6500 spectrofluorotometer. The instrument was programmed to record each spectrum three times and obtain the average; the difference between the individual spectras was less than 0.01% of the average. The values at the peaks corresponding to the maximum of CFP emission (490 nm) and YFP (525 nm) were logged in as “total CFP fluorescence” and “total YFP fluorescence”, respectively. The changes in these values were monitored upon addition of GST-R7DEP, GST or the storage buffer in which the GST or GST-R7DEP stocks were prepared. To study the dose-dependence of this fluorescence change from added GST-R7DEP, the stock solution was consecutively added to the lysate in 50 μl increments. GST stock or the buffer was added in the similar manner to the control aliquots of the lysate. Upon addition of each portion of the GST-R7DEP or GST stock, the recorded fluorescence values were observed to drop by approximately 2.5% due to the dilution of the lysate. To calculate the specific effect of GST-R7DEP, the CFP and YFP fluorescence values determined upon addition of GST (F_{GST}) are subtracted from the values obtained with GST-R7DEP (F_{DEP}). The difference is positive for CFP and negative for YFP. The difference between F_{GST} and F_{DEP} was proportional to the GST-R7DEP concentration. The effect of GST is identical to the effect of the buffer.

R7BP-expressing stable CHO-K1 cells. A stable cell line expressing R7BP (CHO-R7BP) was generated through clonal selection on geneticin. Clones were analyzed by Western blot and six were selected and characterized with respect to recruitment of RGS7 to the membrane. The clone with the highest R7BP expression was used in the experiments.

Ca²⁺ mobilization assay. CHO-K1 or CHO-R7BP cells were transiently transfected, using a standard protocol (7), with cDNAs for M3 muscarinic receptor, RGS7 and Gβ₅, or Lac Z, as required by the experiment. Transfected cells were grown on coverslips and 48 hours later were washed with 2% FBS in HBSS. Cells were the incubated in 2% FBS in HBSS containing 1 μM fura-2AM for 45 minutes at ambient temperature in the dark. This was followed by a 30 minute incubation in Locke's buffer to permit the de-esterification of fura-2AM. The coverslips were then secured in a flow chamber and mounted on the stage of a Nikon TE2000 inverted fluorescence microscope. The cells were continuously perfused with Locke's buffer and stimulated with 100 μM carbachol in the same buffer. The images were collected in real time every two seconds using a 20× UV objective lens and recorded using Metafluor software. The excitation wavelengths were 340 and 380 nm and the emission was set at 510 nm. Free Ca²⁺ concentration was determined from the fluorescence measurements using the fura-2 Ca²⁺ imaging calibration kit (Molecular Probes) according to manufacturer's instructions.

### Example 2

### Obesity Onset in Heterozygote Gβ₅ Mice

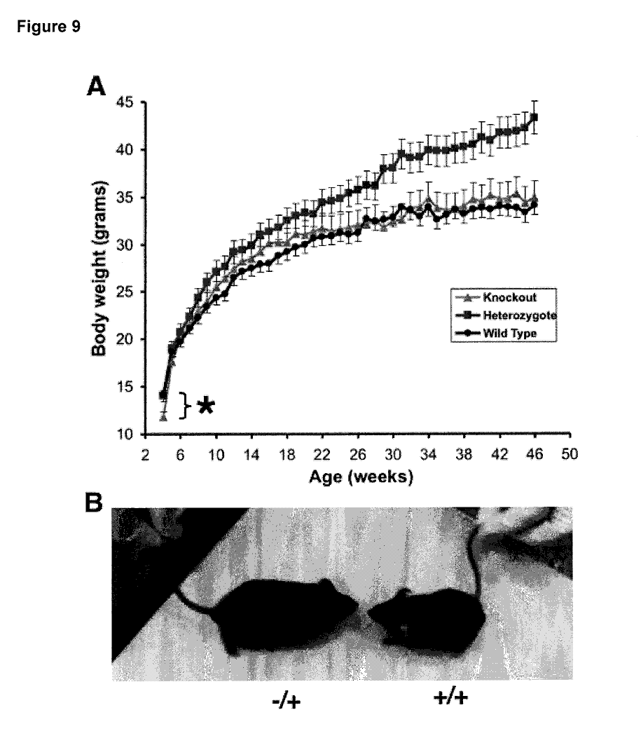

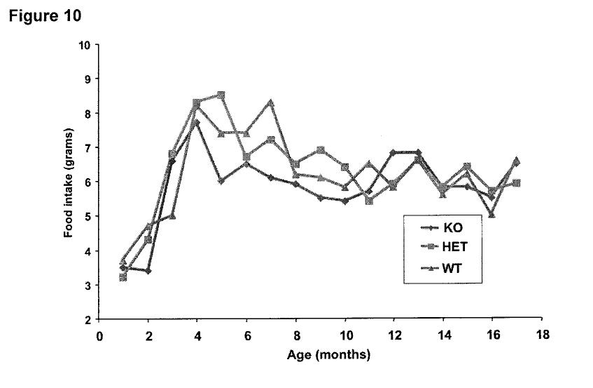

Knockout mice, which lack both copies of Gβ₅ gene, were born significantly smaller than the wild-type animals, but reached the size and weight indistinguishable from the wild-type by the age of two months. Heterozygote Gβ₅ knockout mice were born similar in size and weight to wild-type animals, but by six months of age, showed increased body weight compared to wild-type and homozygote knockout mice. By 8 months of age, the wild-type and homozygote mice weighed approximately 31 g; the weight of heterozygotes was 42 g (FIG. 9). The body weight increase in heterozygote mice occurred primarily due to accumulation of body fat. The relative food consumption (food consumed per body weight) by the Gβ₅ knockout, heterozygote and wild-type mice was identical (FIG. 10).

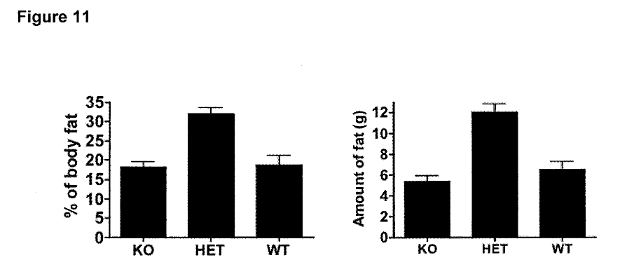

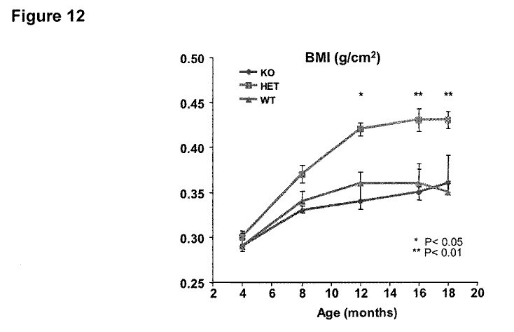

Dual energy X-ray absorptiometry (DEXA) was also used to determine the body fat percentage. The results in FIG. 11 demonstrate that the heterozygote mice have a two-fold increase in fat content. The calculated additional mass of total fat in the heterozygotes was about six grams, which corresponds to the increase in their total body mass (FIG. 9). This indicates that the difference in the BMI (FIG. 12) of the haploinsufficient animals can be ascribed primarily to this additional fat.

To calculate the body mass index (BMI) the values of body weight divided were by squared body length. This demonstrated the age-dependent increase in BMI in heterozygotes as compared to wild-type or knockout age- and sex-matched counterparts mice (FIG. 12), confirming the increased adiposity in the heterozygote animals.

Gβ₅ complex also plays a role in control of moving activity of the mice. The homozygote mice, lacking both copies of Gβ₅ gene, appeared to be hyperactive, whereas the heterozygotes were less active than the wild-type age- and sex-matched counterparts. Similar to the increase in weight, the difference in movement appears to be related to age of the animals. Animals younger than 4 months are indistinguishable with respect to movement pattern or weight.

Methods

Analysis of food weight gain and food intake. The Gβ₅−/− knockout mice were back-crossed four times into the C57BL6 background. Then, they were mated with wild-type mice to produce heterozygote males and females, which in turn were used to obtain age-matched groups of WT, Gβ₅−/+ and Gβ₅−/− mice. Only males were selected for the long-term observation. Three cohorts born two months apart of each other were entered into the experiment. Each cohort consisted of at least four mice each of: WT, Gβ₅−/+ and Gβ₅−/− mice. Cages contained up to five isogenic mice per cage. The weight of mice was recorded once per week. Food consumption was determined as follows: once a week on day one, the weighted amount of regular rodent chow was added to each cage. On day five, the weight of the remaining pellets was determined. The difference in the weight was divided by the number of mice in the cage.

### Example 3

### Gβ5-RGS7 Interacts with Muscarinic M3 Receptor in a Receptor-Selective manner

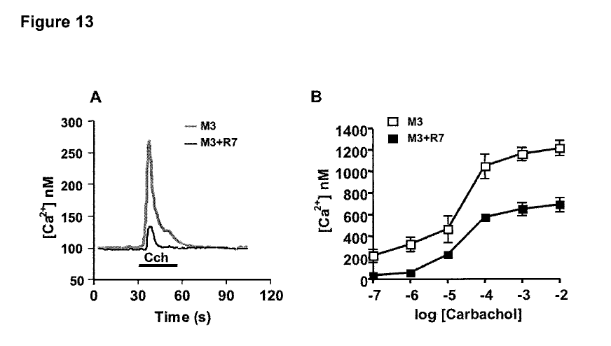

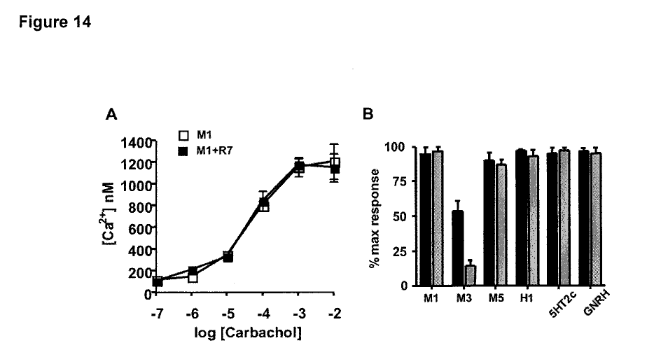

In CHO cells expressing M3R, application of carbachol leads to a robust Ca²⁺ response, and co-expression of Gβ5-RGS7 results in attenuation of this response. While Gβ5-RGS7 had the robust inhibitory effect on M3 receptor, under the same conditions it had no effect on another Gq-coupled receptors, for example muscarinic M1 (FIGS. 13, 14) (22).

If Gβ5-RGS7 acted as the GAP for Gq, it would have inhibited all these GPCRs. Therefore, these results indicate that the inhibition of M3R signal transduction does not involve GAP activity and acted upstream of the G protein. This notion was consistent with previous studies on purified proteins that showed (43, 44) that Gβ5-RGS7 does not possess GAP activity towards Gq. Indeed, inhibition of M3 receptor signaling by Gβ5-RGS7 does not require the RGS domain, but instead, the DEP domain is sufficient to exert the inhibitory action of the entire complex (22).

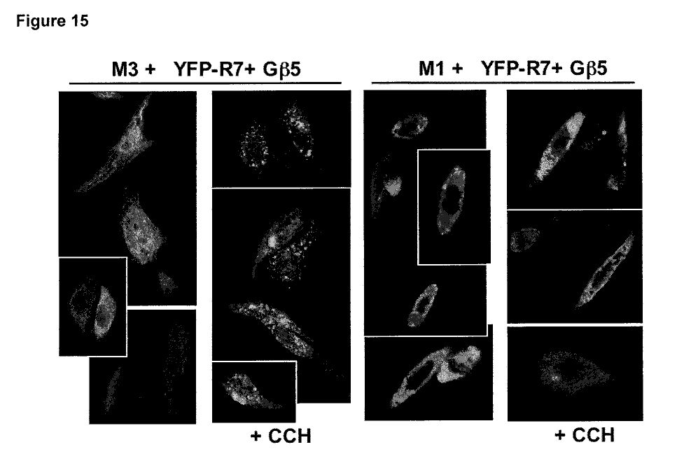

Another aspect of the interaction between the Gβ5-RGS7 complex and M3 receptor is the effect of M3 receptor activation on subcellular localization of Gβ5-RGS7. In CHO cells transfected with Gβ5, RGS7, the complex is found in the cytosol. If these cells are also transfected with M3 receptor, a large portion distributes to endosomes (FIG. 15). Stimulation of M3 receptor with Carbachol (acethylcholine receptor agonist) enhances the distribution of Gβ5-RGS7 to the endosomes, but the presence of nonstimulated M3 receptor was sufficient to do so, presumably because of the basal level of activity of the overexpressed receptor. Like the effect of Gβ5-RGS7 on receptor-mediated Ca²⁺ mobilization, this subcellular distribution is receptor subtype selective. For example, Gβ5-RGS7 remains cytosolic in the presence of muscarinic M1 receptor.

Materials and Methods

Reagents and Antibodies: The cDNA clones for human muscarinic M3, M1, and M5, histamine H1, gonadotropin releasing hormone (GNRH), and serotonin 2c (5HT2c) receptors were obtained from the Missouri S&T cDNA Resource Center (cdna.org). The GFP antibody was from Clontech. RGS7, Gβ5, and Gβ1 antibodies have been described. Carbamoylcholine chloride (carbachol), histamine dihydrochloride, serotonin hydrochloride, and human luteinizing hormone releasing hormone acetate were all obtained from Sigma.

GST-MR i3 Constructs. The GST fusion constructs encoding the third intracellular loops (i3) of M1-5 muscarinic receptors were kindly provided by J. Hepler (Emory University, Atlanta, Ga.) and have been previously described (Bernstein, L. S., et al. (2004) J. Biol. Chem. 279 (20), 21248-21256). The DNA fragment encoding the M3R i3 loop region (Asn³⁰⁴-Gln³⁹⁰) was amplified from the fulllength human M3R using the forward primer 5′-TCCGGATCCAACAGGAGGAAGTAT-3′ (SEQ ID NO: 11) and the reverse primer 5′-CACGAATTCCTGCAGGTTGTCCGA-3′ (SEQ ID NO: 12). The shorter fragment encoding the Ser345-Gln390 part of the i3 loop was amplified using the forward primer 5′-GCCGGTCCTCCCTGGAGAACTCC-3′ (SEQ ID NO: 13) and the reverse primer used for Asn³⁰⁴-Gln³⁹⁰. The fragments were cloned into the pGEX-2T vector at the BamHI and EcoRI sites. The GST fusion constructs encoding rat M3R i3 Arg²⁵²-Gln⁴⁹⁰ and Val³⁹⁰-Gln⁴⁹⁰ (Wu, G., et al., J. Biol. Chem. 273 (13), 7197-7200 (1998); Wu, G., et al. (2000) Biochemistry, Vol. 48, No. 10, (12), 9026-9034) were kindly provided by S. Lanier (Medical University of South Carolina, Charleston, S.C.).

Preparation of Brain Homogenates. Mouse brains were homogenized in lysis buffer [20 mM Tris-HCl (pH 7.5), 1 mM EDTA, 50 mM NaCl, and 2 mM β-mercaptoethanol] and centrifuged at 150000 g for 1.5 h at 4° C. The supernatant fraction was collected (cytosolic extract), and the pellet containing the membranes was washed twice and resuspended in the same buffer containing 1% sodium cholate. This suspension was left on ice for 15 min then centrifuged at 150000 g for 45 min at 4° C., and the supernatant was retained as the membrane extract.

Purification of GST Fusion Proteins. The purification of the GST fusion proteins was done as previously described (Narayanan, V., et al. (2007) Biochemistry 46 (23), 6859-6870). Briefly, bacterial cultures were grown at 37° C., and protein expression was induced with the addition of 0.4 mM IPTG for 1-1.5 h at 37° C. The cultures were harvested and centrifuged, and pellets were resuspended in STE [150 mM NaCl, 100 mM Tris (pH 8.0), and 1 mM EDTA] buffer containing lysozyme, 5 mM DTT, and protease inhibitors. The cell suspension was briefly sonicated on ice, and Sarkosyl (final concentration of 1.5%) and Triton X-100 (final concentration of 2%) were added to the lysate. After gentle rotation at 4° C. for 1 h, the lysate was centrifuged at 19000 rpm at 4° C. for 30 min. The clarified lysate was batch processed using GST-Sepharose 4B beads (GE) (0.5 mL of packed beads per 10 mL of lysate, which contained 2-5 mg/mL total protein) overnight at 4° C. The beads were washed and eluted with 20 mM glutathione. The purified GST fusion protein was desalted on Sephadex G-25 preequilibrated with buffer containing 100 mM Tris (pH 8.0), 150 mM NaCl, and 15% glycerol and stored frozen in aliquots at −80° C.

Constructs for Expression in Mammalian Cells. The plasmid harboring the M3R-short receptor has 196 amino acids deleted from the i3 loop (amino acids Ala274-Lys469), provided. To generate the construct corresponding to amino acids 1-124 of full-length bovine RGS7 (YFP-DEP), nucleotides 1-372 were amplified using the forward primer 5′-TCCGGACTCAGATCTATGGCCCAGGGG-3′ (SEQ ID NO: 14) and the reverse primer 5′-GTCTGTGTTAAGCTTTTCCGGCTCCCA-3′ (SEQ ID NO: 15). The RGS7 construct that lacks the RGS domain and the C-terminus (ΔRGS) was generated by PCR amplification of nucleotides 1-963 corresponding to amino acids 1-321 using the forward primer 5′-TCCGGACTCAGATCTATGGCCCAGGGG-3′ (SEQ ID NO: 16) and the reverse primer 5′-CCCAAGCTTTTCTTTGCTTGC-3′ (SEQ ID NO: 17). These fragments were cloned into the pEYFPC1 vector at BglII and HindIII sites. The constructs encoding the C-terminal part of RGS7 (RGS7249-469), YFP-RGS7, and CFP-fused Gβ5 were described previously (Narayanan, V et al. (2007) Biochemistry 46 (23), 6859-6870; Witherow, D. S., et al (2003) J. Biol. Chem. 278 (23), 21307-21313).

Cell Culture and Transfection. CHO-K1 cells were cultured in F-12K Nutrient Mixture (Kaighn's modification, Gibco) with 10% fetal bovine serum and penicillin/streptomycin. CHO-R7BP cells were cultured like the CHOK1 cells with the addition of 400 μg/mL Geneticin. Twenty four hours prior to transfection, the cells were plated to achieve a density of 0.8-1.0×10⁶ cells per 100 mm plate. Transfection was carried out using Lipofectamine 2000 (Invitrogen) as per the manufacturer's instructions. The DNA ratio of RGS7 to Gβ5 was maintained at 5:1, with a total of 8.0 μg of DNA per plate. Lac Z DNA was used as a control to ensure that the total DNA per plate used in the CHO-K1 cotransfection assays remained constant. Forty-eight hours after transfection, cells were washed with HBSS, harvested, lysed in hypotonic buffer, and used for pull-down assays.

GST Pull Down. Glutathione Sepharose 4B beads were prewashed with PBS and 0.1% CHAPS, incubated at 4° C. with purified recombinant GST or the GST fusion proteins for 1-2 h, and then washed three times with PBS and 0.1% CHAPS to remove excess protein. The slurry was incubated overnight at 4° C. on a rotary mixer with the investigated CHO cell lysates, as determined by the experiment. At the end of the incubation, the beads were settled by gravity and the supernatant was collected as the unbound fraction. In a typical assay, the packed volume of the resin was 30 μL, and the volume of the protein lysate was 300 μL. The beads were washed three times with 600 μL of PBS and 0.1% CHAPS buffer and eluted with 30 μL of 2×SDS sample loading buffer. The unbound and eluted fractions were resolved by gel electrophoresis and analyzed by Western blotting.

Ca²⁺ Mobilization Assay. CHO-K1 cells were transiently transfected with cDNAs for M1, M3, M5, GNRH, H1, and 5HT2c receptors, RGS7 and Gβ5, or Lac Z, as required by the experiment. Transfected cells were grown on 12 mm glass coverslips (Electron Microscopy Sciences). Forty-eight hours after transfection, they were washed with 2% FBS in HBSS and then incubated in 2% FBS in HBSS containing 1 μM fura-2AM for 45 min at ambient temperature in the dark. This was followed by a 30 min incubation in Locke's buffer to permit de-esterification of fura-2AM. The coverslips were then secured in a flow chamber and mounted on the stage of a Nikon TE2000 inverted fluorescence microscope. The cells were continuously perfused with Locke's buffer and stimulated with varying agonist concentrations in the same buffer as required by the experiment. The images were collected in real time every 2 s using a 20× UV objective lens and recorded using Metafluor. The excitation wavelengths were 340 and 380 nm, and the emission was set at 510 nm. The free Ca²⁺ concentration was determined from the fluorescence measurements using the fura-2 Ca²⁺ imaging calibration kit (Molecular Probes) according to the manufacturer's instructions.

[³H]NMS Binding Assay. Muscarinic receptor density was determined by the ligand binding assay using the muscarinic antagonist N-methyl scopolamine chloride ([³H]NMS, 70 Ci/mmol, Perkin-Elmer). Briefly, CHO-K1 cells were transfected in 24-well plates with the M1, M3, or M5 muscarinic receptor alone or together with the Gβ5-RGS7 complex, as required by the experiment. Twenty-four hours after transfection, cells were washed and incubated with 1 mL of [³H]NMS in a buffer also containing 10 mM HEPES (pH 7.4), 4.2 mM NaHCO₃, 11.7 mM glucose, 1.2 mM MgSO₄, 1.2 mM KH₂PO₄, 4.7 mM KCl, 118 mM NaCl, and 1.3 mM CaCl₂. The [³H]NMS concentrations ranged from 20 pM to 14 nM, with seven points used for the saturation curve and Scatchard analysis. Each [³H]NMS concentration was used in duplicate (two wells of cells). Nonspecific binding was assessed in the presence of 20 μM atropine. Following incubation for 1 h at 37° C., cells were rapidly washed twice with 1 mL of ice cold buffer and then lysed with 0.5 mL of 0.1 M NaOH added to the wells. This lysate was neutralized with 0.5 mL of 0.1 M HCl, and the mixture was transferred to the vials for liquid scintillation counting.

Results:

The Gβ5-RGS7 Complex Inhibits M3R-Mediated Ca²⁺ Mobilization. The Gβ5-RGS7 complex attenuated Ca²⁺ mobilization elicited by the muscarinic acetylcholine M3 receptor (M3R) by approximately 50% (Witherow, D. S., et al. (2003) J. Biol. Chem. 278 (23), 21307-21313). Here, it was tested if this inhibition could occur to a greater extent at a lower agonist concentration. It was reasoned that given the high expression level of the receptor in the transiently transfected cells, the amount of Gβ5-RGS7 complex was insufficient to quench the receptor-mediated activation of Gq, particularly at saturating agonist concentrations. To reduce the amount of activated receptor, cells were stimulated with a range of carbachol concentrations. It was found that at carbachol concentrations of 1 and 0.1 μM (below the EC₅₀), the Gβ5-RGS7 complex-mediated inhibition of Ca²⁺ responses was nearly complete.

Receptor Selectivity of Gβ5-RGS7. While Gβ5-RGS7 displayed a robust inhibitory effect on M3R, it had no effect on other tested Gq-coupled receptors. M1R was insensitive to Gβ5-RGS7 over the wide range of tested agonist concentrations. Likewise, the receptors for histamine (H1), serotonin (5HT2c), GNRH, and muscarinic acetylcholine receptor M5R were also not affected by Gβ5-RGS7. The number of binding sites were determined on live CHO-K1 cells expressing M1, M3, and [³H]NMS. The binding of [3H]NMS was saturable and dose dependent, and the Bₘₐₓ values in M1R, M3R, and M5R transfected cells were as follows: 856±81.4 (n=3), 1066±187.9 (n=4), and 1024±81.5 (n=2) fmol/mg of total protein, respectively. The determined K_{d} values for [³H]NMS were 0.310 (0.06, 0.228 (0.09, and 0.401 (0.02 nM, respectively. It was found that the co-expression of Gβ5-RGS7 slightly increased the Bₘₐₓ of all three tested receptors [985±118.0 (n=3), 1310±135.2 (n=4), and 1304±153.0 (n=2) fmol/mg, respectively], which can likely be attributed to a minor positive effect on the transfection efficiency of the CHO cells. The K_{d} values in the presence of Gβ5-RGS7 were 0.277±0.08, 0.216±0.04, and 0.392±0.06 nM, respectively, not appreciably different from the values in the absence of Gβ5-RGS7. Thus, all three muscarinic receptors were expressed at approximately the same level with or without Gβ5-RGS7, however, Gβ5-RGS7 inhibited only signaling elicited by the M3 receptor subtype. Since all tested receptors act through Gq, such receptor selectivity indicated that inhibition of M3R signal transduction occurs upstream of the G protein. Therefore, it was hypothesized that the Gβ5-RGS7 complex inhibited M3R signal transduction via a mechanism that does not utilize its GAP activity.

The DEP Domain of RGS7Is Responsible for M3R Inhibition. To determine the mechanism by which Gβ5-RGS7 inhibits M3R signaling, it was investigated which domain of RGS7 was responsible for this effect. The following three constructs were prepared: (1) the N-terminal portion of RGS7, which lacked the RGS domain and the C-terminus, termed ΔRGS, (2) the C-terminal portion that lacked the N-terminus and the DEP and DHEX domains, termed RGS7249-469, and (3) the N-terminal portion (amino acids 1-124) termed the DEP domain. The ΔRGS and the DEP domain constructs were fused to the C-terminus of YFP to enhance their expression and detection. The ΔRGS and RGS7249-469 truncations of RGS7 were coexpressed in CHO cells together with Gβ5. Our results showed that the ΔRGS and DEP constructs inhibited M3R-induced Ca²⁺ mobilization in a manner similar to that of the full-length RGS7 protein. In contrast, RGS7249-469, which lacked the DEP domain, had no effect on M3R signaling. These results indicated that the RGS domain is not essential for this inhibition, supporting the hypothesis that Gβ5-RGS7 inhibits M3R-mediated signal transduction via a non-GAP mechanism. Instead, the Gβ5-RGS7 complex inhibits M3R signaling via the DEP domain. Similar to full-length Gβ-RGS7, the DEP domain did not inhibit signaling from the M1R, showing the selectivity of the DEP domain of RGS7 toward the M3 receptor subtype.

The DEP Domain of RGS7 Directly Binds to the Third Loop of M3R. The selectivity of Gβ5-RGS7 toward M3R indicated that it acts upstream of the G protein, suggesting that it may directly bind to the receptor. The i1 and i2 loops are very short and well-conserved among all five muscarinic receptors. In contrast, the i3 loops of muscarinic receptors are longer, are much more diverse, and interact with multiple proteins such as GRq, Gβγ, β-arrestin, RGS2, calmodulin, and SET. Therefore, it was reasoned that the likely binding site for Gβ5-RGS7 could be located within the third intracellular loop. To test this hypothesis, it was first investigated if Gβ5-RGS7 had an effect on M3R-short, the deletion mutant of M3R that lacked a large portion of the i3 loop. The results showed that Gβ5-RGS7 had no effect on Ca²⁺ mobilization elicited by this M3R mutant. The hypothesis that the DEP binding site is localized within the i3 loop was further tested using a pull-down assay with the i3 loop of M3R fused to the C-terminus of GST. It was found that the DEP domain of RGS7 bound to the M3R i3 loop, but not to GST. The DEP domain also bound to the i3 loop of M5R and also exhibited very weak binding to the loops of M1 and M2 receptors. To locate the putative binding site for the DEP domain, its interaction with shorter fragments of the M3R i3 loop was tested. The results showed that the DEP domain binds to the region encompassing amino acids 345-390 in human M3R, corresponding to the central portion of the i3 loop. Thus, it appears that RGS7 DEP binds to the region that is most distant from the membrane, whereas the reported binding sites for the G protein are located at the ends of the third loop, presumably near the membrane surface.

Effects of Gβ5 and R7BP on the Interaction of RGS7 with M3R. It was found that full-length monomeric RGS7 bound to the i3 loop of M3R, but the Gβ5-RGS7 dimer did not. Likewise, Gβ5 blocked the interaction of the ΔRGS construct with the M3R i3 loop. Neither the Gβ5-RGS7²⁴⁹⁻⁴⁶⁹ complex nor monomeric Gβ5 bound to the i3 loop. The effects of R7BP on the interaction of Gβ5-RGS7 with M3R were investigated. The results showed that R7BP prevented the interaction of the DEP domain or the full-length monomeric RGS7 with the i3 loop of M3R. This result is consistent with the observation that in CHO cells stably expressing R7BP, Gβ5-RGS7 did not influence M3R-mediated Ca²⁺ mobilization. Gβ5-RGS7 in the native tissue was present in both membranes and cytosol, whereas R7BP was found only in the membranes. These results indicate that binding of DEP to the i3 loop and binding to R7BP are mutually exclusive and indicate that the regulation of M3R is carried out by the cytosolic form of Gβ5-RGS7.

### DISCUSSION

The function of a neuronal regulator of G protein signaling, the Gβ5-RGS7 complex was investigated. This study highlights two novel aspects: the remarkable selectivity of Gβ5-RGS7 toward muscarinic M3 receptors (M3R) and the direct interaction of the DEP domain of RGS7 with the receptor.

Receptor Selectivity. The experiments showed that Gβ5-RGS7 robustly inhibited signaling from M3R, but under the same experimental conditions, it did not influence other Gq-coupled receptors, including M1R. This selectivity toward M3R indicated that Gβ5-RGS7 inhibits M3R signaling not via the GAP activity toward Gq, but upstream of the G protein. It was hypothesized that Gβ5-RGS7 interacts directly with the receptor. Since it was shown that M3R contains the binding site for Gβγ subunits, it was initially thought that the Gβ5/GGL moiety was responsible for this interaction. However, the results showed that the effect of Gβ5-RGS7 was mediated by the DEP domain, whereas neither Gβ5/GGL nor the RGS domain was needed for the inhibition of M3R. Previous studies showed that Gβ5-RGS7 has GAP activity toward Gi but not Gq family G proteins. Therefore, it was not clear why Gβ5-RGS7 inhibited Ca²⁺ mobilization elicited by the Gq-coupled M3R. The current findings showed that this inhibition is mediated by the interaction between the receptor and the DEP domain of RGS7. The identification of this novel mechanism resolved the controversy between the lack of GAP activity toward GRq and the functional effect of Gβ5-RGS7 on the M3R in cells.

The M3R is expressed in a variety of peripheral tissues as well as in the central nervous system. According to the Allen Brain Atlas (brain-map.org), M3R and RGS7 mRNAs co-localize, especially in the cerebral cortex and hippocampus. Interestingly, muscarinic receptors of different subtypes can be found in the same neurons. It is not clear why two Gq-coupled receptors of acetylcholine are needed in one cell. It is reasonable to hypothesize that the interaction with Gβ5-RGS7 differentiates the neuronal M3R from the M1R and from the M3R expressed in peripheral cells. Mice lacking the neuronal M3R have a distinct lean phenotype, which is different from the phenotypes of other muscarinic receptor knockouts. The Gβ5 knockout mice are born runty and remain lean throughout their lifetime, even on a high-fat diet. The similarity in the phenotypes of Gβ5 and neuronal M3R knockouts may indicate that they participate in the same pathway that is unique for neuronal signaling.

New Role of the DEP Domain. It was found that the isolated DEP domain of RGS7 mimicked the functional effect of the entire RGS7 complex, supporting the concept that RGS proteins can regulate signal transduction not only via their GAP activity. The results herein, show that the DEP domain of RGS7 can directly bind to the i3 loop of M3R, which contains binding sites for several other proteins, including Gαq, Gβγ, arrestin, calmodulin, and SET. In contrast to Gαq, RGS7 binds to the middle of the loop, which can potentially protrude far into the cytosol. Without wishing to be bound by theory, it is speculated that the distance of approximately 100 amino acids between the RGS7 site and the juxtamembrane site for Gq could allow the cytosolic Gβ5-RGS7 dimer to bind to the receptor at the same time with Gαq. This would be consistent with results that detected FRET between the fluorescently tagged Gβ5-RGS7 complex and Gαq in cells.

The RGS7 binding site partially encompasses the region phosphorylated by GRK2 and casein kinase and a binding site for α-arrestin, which suggests that Gβ5-RGS7 could have a role in the processes of M3R desensitization and α-arrestin-mediated signaling. The results showed that binding of the RGS7 DEP domain to the isolated i3 loops of muscarinic receptors had lower subtype selectivity than the inhibitory effect on the full-length receptors. Binding to the M3R i3 loop was the most robust, but the DEP domain also bound well to the M5R i3 loop and exhibited much weaker interaction with the i3 loops of M1 and M2 receptors. The reason for the reduced selectivity in the GST pulldown assay compared to the Ca²⁺ mobilization experiments is not clear at this point. One can speculate that the size of the M3R i3 loop allows the RGS7 complex to remain associated once the G protein binds to the juxta-membrane regions and thus be more effective in inhibiting Gq activation. It is also possible that there is an additional site in the full-length M3R that stabilizes the interaction with the DEP domain and which is absent in other receptors.

It was recently shown that the DEP domain of the yeast RGS protein Sst2 could directly bind to the G protein-coupled receptor, Step 2. That interaction occurred at the C-terminal tail of Step 2, which is different from the RGS7-M3R interaction. There is no obvious homology between the C-tail of Step 2 and the i3 loop of M3R; however, both these regions contain the sites for phosphorylation and play a role in desensitization. Another study showed that the dopamine D2 receptor facilitated the membrane localization of RGS9-2, a member of the R7 family of RGS proteins. Like the interaction of M3R with RGS7 reported herein, the effect of the dopamine receptor was selective for the D2 subtype and was mediated by the DEP domain of RGS9-2. However, it did not require the third loop or the C-terminus of the D2 receptor, indicating that structural elements involved in the interaction with DEP domains can be different for specific receptor subtypes. It is worth noting that another DEP domain-containing signaling protein, Disheveled, also binds to its cognate seven-pass transmembrane receptor, Frizzled. However, this interaction is mediated by its PDZ domain rather than the DEP domain of Disheveled. It will be interesting to determine whether the DEP domains found in RGS proteins are unique in their ability to interact with GPCRs.

Potential Roles of Gβ5 and R7BP. G protein β subunit Gβ5 interacts with RGS proteins of the R7 family instead of Gγ subunits. Gβ5 and the associated RGS protein stabilize each other against rapid proteolysis. This mutual stabilization explained why R7 proteins and Gβ5 have not been found apart from each other in the native tissues and why R7 proteins are absent in Gβ5 knockout animals. The knockout of Gβ5 also causes the disappearance of R7BP. However, it is not clear why such basic function as stabilization of the RGS protein would require association with a G protein β subunit. It appears that there must also be a functional role for Gβ5 within the R7 complex. Gβ5 may attenuate the interaction of RGS7 with Gαo. The data herein, show that Gβ5 can prevent the protein-protein interaction between the DEP domain and the i3 loop of the M3 receptor. Thus, it appears that Gβ5 can serve as the negative regulator of both the DEP and RGS domains of RGS7. We found that like Gβ5, R7BP prevents the DEP domain from binding to the i3 loop of M3R. R7BP also blocks the effect of the Gβ5-RGS7 complex on M3R signaling. Therefore, R7BP and Gβ5 differ in their effects on the interaction of RGS7 with the full-length M3R in intact cells. The Gβ5-RGS7 complex is as effective as the isolated DEP domain of RGS7 in its ability to block M3R signaling under the same experimental conditions. This indicates that Gβ5 can allow the DEP domain to interact with the i3 loop protruding from the full-length receptor in intact cells.

On the basis of the results presented herein, it is hypothesized that the agonist-bound M3R can open the cytosolic Gβ5-RGS7 dimer so that the DEP domain binds to the i3 loop, which then inhibits M3R-Gq coupling. The interaction with R7BP at the membrane restricts the action of Gβ5-RGS7 to Gi-coupled receptors, such as muscarinic M2, which occurs via the canonical GAP mechanism that involves the RGS domain of RGS7.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected. In addition, all references cited herein are indicative of the level of skill in the art and are hereby incorporated by reference in their entirety.

### REFERENCES

1. Siderovski, D. P., Strockbine, B., and Behe, C. I., Whither goest the RGS proteins?, Crit. Rev Biochem Mol Biol, 34, 215-251 (1999).
2. Hepler, J. R., Emerging roles for RGS proteins in cell signalling, Trends Pharmacol Sci, 20, 376-382 (1999).
3. Grabowska, D., Jayaraman, M., Kaltenbronn, K. M., Sandiford, S. L., Wang, Q., Jenkins, S., Slepak, V. Z., Smith, Y., and Blumer, K. J., Postnatal induction and localization of R7BP, a membrane-anchoring protein for regulator of G protein signaling 7 family-Gbeta5 complexes in brain, Neuroscience, 151, 969-982 (2008).
4. Khawaja, X. Z., Liang, J. J., Saugstad, J. A., Jones, P. G., Harnish, S., Conn, P. J., and Cockett, M. I., Immunohistochemical distribution of RGS7 protein and cellular selectivity in colocalizing with Galphaq proteins in the adult rat brain, J Neurochem, 72, 174-184 (1999).
5. Elmore, T., Rodriguez, A., and Smith, D. P., dRGS7 encodes a Drosophila homolog of EGL-10 and vertebrate RGS7, DNA Cell Biol, 17, 983-989 (1998).
6. Gold, S. J., Ni, Y. G., Dohlman, H. G., and Nestler, E. J., Regulators of G-protein signaling (RGS) proteins: region-specific expression of nine subtypes in rat brain, J Neurosci, 17, 8024-8037 (1997).
7. Cabrera, J. L., de Freitas, F., Satpaev, D. K., and Slepak, V. Z., Identification of the Gbeta5-RGS7 protein complex in the retina, Biochem Biophys Res Commun, 249, 898-902 (1998).
8. Witherow, D. S., Wang, Q., Levay, K., Cabrera, J. L., Chen, J., Willars, G. B., and Slepak, V. Z., Complexes of the G protein subunit gbeta 5 with the regulators of G protein signaling RGS7 and RGS9. Characterization in native tissues and in transfected cells, J Biol Chem, 275, 24872-24880 (2000).
9. Zhang, J. H., and Simonds, W. F., Copurification of brain G-protein beta5 with RGS6 and RGS7, J Neurosci, 20, RC59 (2000).
10. Watson, A. J., Katz, A., and Simon, M. I., A fifth member of the mammalian G-protein beta-subunit family. Expression in brain and activation of the beta 2 isotype of phospholipase C, J Biol Chem, 269, 22150-22156 (1994).
11. Liang, J. J., Cockett, M., and Khawaja, X. Z., Immunohistochemical localization of G protein beta1, beta2, beta3, beta4, beta5, and gamma3 subunits in the adult rat brain, J Neurochem, 71, 345-355 (1998).
12. Witherow, D. S., and Slepak, V. Z., A Novel Kind of G Protein Heterodimer: The Gbeta5-RGS Complex, Receptors Channels, 9, 205-212 (2003).
13. Snow, B. E., Krumins, A. M., Brothers, G. M., Lee, S. F., Wall, M. A., Chung, S., Mangion, J., Arya, S., Gilman, A. G., and Siderovski, D. P., A G protein gamma subunit-like domain shared between RGS11 and other RGS proteins specifies binding to Gbeta5 subunits, Proc Natl Acad Sci USA, 95, 13307-13312 (1998).
14. Levay, K., Cabrera, J. L., Satpaev, D. K., and Slepak, V. Z., Gbeta5 prevents the RGS7-Galphao interaction through binding to a distinct Ggamma-like domain found in RGS7 and other RGS proteins, Proc Natl Acad Sci USA, 96, 2503-2507 (1999).
15. Snow, B. E., Betts, L., Mangion, J., Sondek, J., and Siderovski, D. P., Fidelity of G protein beta-subunit association by the G protein gamma-subunit-like domains of RGS6, RGS7, and RGS11, Proc Natl Acad Sci USA, 96, 6489-6494 (1999).
16. Sondek, J., and Siderovski, D. P., Ggamma-like (GGL) domains: new frontiers in G-protein signaling and beta-propeller scaffolding, Biochem Pharmacol, 61, 1329-1337 (2001).
17. Jones, M. B., Siderovski, D. P., and Hooks, S. B., The G{beta} {gamma} DIMER as a NOVEL SOURCE of SELECTIVITY in G-Protein Signaling: GGL-ing AT CONVENTION, Mol Interv, 4, 200-214 (2004).
18. Chen, C. K., Burns, M. E., He, W., Wensel, T. G., Baylor, D. A., and Simon, M. I., Slowed recovery of rod photoresponse in mice lacking the GTPase accelerating protein RGS9-1, Nature, 403, 557-560 (2000).
19. Chen, C. K., Eversole-Cire, P., Zhang, H., Mancino, V., Chen, Y. J., He, W., Wensel, T. G., and Simon, M. I., Instability of GGL domain-containing RGS proteins in mice lacking the G protein beta-subunit Gbeta5, Proc Natl Acad Sci USA, 100, 6604-6609 (2003).
20. Chase, D. L., Patikoglou, G. A., and Koelle, M. R., Two RGS proteins that inhibit Galpha(o) and Galpha(q) signaling in C. elegans neurons require a Gbeta(5)-like subunit for function, Curr Biol, 11, 222-231 (2001).
21. Narayanan, V., Sandiford, S. L., Wang, Q., Keren-Raifman, T., Levay, K., and Slepak, V. Z., Intramolecular Interaction between the DEP Domain of RGS7 and the Gbeta(5) Subunit, Biochemistry, 46, 6859-6870 (2007).
22. Sandiford, S., and Slepak, V., G5-RGS7 selectively inhibits muscarinic M3 receptor signaling via the interaction between the third intracellular loop of the receptor and the DEP domain of RGS7, Biochemistry, 48, 2282-2289 (2009).
23. Cone, R. D., Anatomy and regulation of the central melanocortin system, Nat Neurosci, 8, 571-578 (2005).
24. Kojima, M., Hosoda, H., Date, Y., Nakazato, M., Matsuo, H., and Kangawa, K., Ghrelin is a growth-hormone-releasing acylated peptide from stomach, Nature, 402, 656-660 (1999).
25. Katoh, K., Kuma, K., Toh, H., and Miyata, T., MAFFT version 5: improvement in accuracy of multiple sequence alignment, Nucleic Acids Res, 33, 511-518 (2005).
26. Katoh, K., Misawa, K., Kuma, K., and Miyata, T., MAFFT: a novel method for rapid multiple sequence alignment based on fast Fourier transform, Nucleic Acids Res, 30, 3059-3066 (2002).
27. Gouet, P., Courcelle, E., Stuart, D. I., and Metoz, F., ESPript: analysis of multiple sequence alignments in PostScript, Bioinformatics, 15, 305-308 (1999).
28. Witherow, D. S., Tovey, S. C., Wang, Q., Willars, G. B., and Slepak, V. Z., G beta 5.RGS7 inhibits G alpha q-mediated signaling via a direct protein-protein interaction, J Biol Chem, 278, 21307-21313 (2003).
29. Drenan, R. M., Doupnik, C. A., Boyle, M. P., Muglia, L. J., Huettner, J. E., Linder, M. E., and Blumer, K. J., Palmitoylation regulates plasma membrane-nuclear shuttling of R7BP, a novel membrane anchor for the RGS7 family, J Cell Biol, 169, 623-633 (2005).
30. Bonacci, T. M., Mathews, J. L., Yuan, C., Lehmann, D. M., Malik, S., Wu, D., Font, J. L., Bidlack, J. M., and Smrcka, A. V., Differential targeting of Gbetagamma-subunit signaling with small molecules, Science, 312, 443-446 (2006).
31. Wong, H. C., Mao, J., Nguyen, J. T., Srinivas, S., Zhang, W., Liu, B., Li, L., Wu, D., and Zheng, J., Structural basis of the recognition of the dishevelled DEP domain in the Wnt signaling pathway, Nat Struct Biol, 7, 1178-1184 (2000).
32. Rose, J. J., Taylor, J. B., Shi, J., Cockett, M. I., Jones, P. G., and Hepler, J. R., RGS7 is palmitoylated and exists as biochemically distinct forms, J Neurochem, 75, 2103-2112 (2000).
33. Kovoor, A., Chen, C. K., He, W., Wensel, T. G., Simon, M. I., and Lester, H. A., Co-expression of Gbeta5 enhances the function of two Gamma subunit-like domain-containing regulators of G protein signaling proteins, J Biol Chem, 275, 3397-3402 (2000).
34. Keren-Raifman, T., Bera, A. K., Zveig, D., Peleg, S., Witherow, D. S., Slepak, V. Z., and Dascal, N., Expression levels of RGS7 and RGS4 proteins determine the mode of regulation of the G protein-activated K(+) channel and control regulation of RGS7 by G beta 5, FEBS Lett, 492, 20-28 (2001).
35. Skiba, N. P., Martemyanov, K. A., Elfenbein, A., Hopp, J. A., Bohm, A., Simonds, W. F., and Arshaysky, V. Y., RGS9-G beta 5 substrate selectivity in photoreceptors. Opposing effects of constituent domains yield high affinity of RGS interaction with the G protein-effector complex, J Biol Chem, 276, 37365-37372 (2001).
36. He, W., Lu, L., Zhang, X., El-Hodiri, H. M., Chen, C. K., Slep, K. C., Simon, M. I., Jamrich, M., and Wensel, T. G., Modules in the photoreceptor RGS9-1.Gbeta 5L GTPase-accelerating protein complex control effector coupling, GTPase acceleration, protein folding, and stability, J Biol Chem, 275, 37093-37100 (2000).
37. Martemyanov, K. A., and Arshaysky, V. Y., Noncatalytic domains of RGS9-1.Gbeta 5L play a decisive role in establishing its substrate specificity, J Biol Chem, 277, 32843-32848 (2002).
38. Zhang, J. H., Barr, V. A., Mo, Y., Rojkova, A. M., Liu, S., and Simonds, W. F., Nuclear localization of G protein beta 5 and regulator of G protein signaling 7 in neurons and brain, J Biol Chem, 276, 10284-10289 (2001).
39. Rojkova, A. M., Woodard, G. E., Huang, T. C., Combs, C. A., Zhang, J. H., and Simonds, W. F., Ggamma subunit-selective G protein beta 5 mutant defines regulators of G protein signaling protein binding requirement for nuclear localization, J Biol Chem, 278, 12507-12512 (2003).
40. Hunt, R. A., Edris, W., Chanda, P. K., Nieuwenhuijsen, B., and Young, K. H., Snapin interacts with the N-terminus of regulator of G protein signaling 7, Biochem Biophys Res Commun, 303, 594-599 (2003).
41. Gerachshenko, T., Blackmer, T., Yoon, E. J., Bartleson, C., Hamm, H. E., and Alford, S., Gbetagamma acts at the C terminus of SNAP-25 to mediate presynaptic inhibition, Nat Neurosci, 8, 597-605 (2005).
42. Blackmer, T., Larsen, E. C., Bartleson, C., Kowalchyk, J. A., Yoon, E. J., Preininger, A. M., Alford, S., Hamm, H. E., and Martin, T. F., G protein betagamma directly regulates SNARE protein fusion machinery for secretory granule exocytosis, Nat Neurosci, 8, 421-425 (2005).
43. Hooks, S. B., Waldo, G. L., Corbitt, J., Bodor, E. T., Krumins, A. M., and Harden, T. K., RGS6, RGS7, RGS9, and RGS11 stimulate GTPase activity of Gi family G-proteins with differential selectivity and maximal activity, J Biol Chem, 278, 10087-10093 (2003).
44. Posner, B. A., Gilman, A. G., and Harris, B. A., Regulators of G protein signaling 6 and 7. Purification of complexes with gbeta5 and assessment of their effects on g protein-mediated signaling pathways, J Biol Chem, 274, 31087-31093 (1999).

We claim:

1. A method of identifying a compound capable of modulating the conformation of Gβ₅ complex, comprising determining the interaction between a first Gβ₅ complex fusion protein and a second Gβ₅ complex fusion protein and comparing the determined interaction in the presence and absence of a test compound, wherein a difference in the determined interaction identifies that the compound is capable of modulating the conformation of Gβ₅ complex.

2. The method of claim 1, wherein a proximity-based assay is used to determine the interaction between the first and second Gβ₅ complex fusion proteins.

3. The method of claim 2, wherein a FRET-based assay is used to detect the interaction between the first and second Gβ₅ complex fusion proteins.

4. The method of claim 2, wherein a BRET-based assay is used to detect the interaction between the first and second Gβ₅ complex fusion proteins.

5. The method of claim 1, wherein an affinity chromatography based assay is used to determine the interaction between the first and second Gβ₅ complex fusion proteins.

6. The method of claim 1, wherein a calcium mobilization based assay is used to determine the interaction between the first and second Gβ₅ complex fusion proteins.

7. The method of claim 1, wherein the compound induces the open conformation of Gβ₅ complex.

8. The method of claim 1, wherein the compound induces the closed conformation of Gβ₅ complex.

9. The method of claim 1, wherein the compound is an agonist of Gβ₅ complex activity.

10. The method of claim 1, wherein the compound is an antagonist of Gβ₅ complex activity.

11. The method of claim 1, wherein at least one Gβ₅ complex fusion protein comprises an RGS protein or a homolog, chimeric protein or derivative thereof.

12. The method of claim 1, wherein at least one Gβ₅ complex fusion protein comprises a Gβ₅ subunit or derivative thereof.

13. The method of claim 11, wherein the RGS protein or homolog, chimeric protein or derivative thereof is a protein capable of association with Gβ₅.

14. The method of claim 13, wherein the RGS protein is selected from the group consisting of RGS6, RGS7, RGS9, RGS11.

15. The method of claim 1, wherein Gβ₅ complex activity is associated with obesity.

16. The method of claim 1, wherein the compound is selected from a library of compounds.

17. The method of claim 1, wherein the method is a high throughput screening method.

18. A compound capable of modulating conformation of a Gβ₅ complex identified by a method comprising: determining the interaction between a first Gβ₅ complex fusion protein and a second Gβ₅ complex fusion protein and comparing the determined interaction in the presence and absence of a test compound, wherein a difference in the determined interaction identifies that the compound is capable of modulating the conformation of Gβ₅ complex.

19. The compound of claim 18, wherein the identified compound is administered to a patient in a pharmaceutically acceptable carrier or excipient.

20. A method of treating a disorder associated with Gβ₅ complex activity in an individual in need thereof comprising administering an effective amount of a composition comprising a compound which modulates conformation of the Gβ₅ complex in a pharmaceutically acceptable carrier or excipient.

21. The method of claim 20, wherein the disorder is obesity.

22. The method of claim 20, wherein the disorder is a neurological disorder.

23. A recombinant protein comprising Gβ₅ complex in an open conformation.

24. The recombinant protein of claim 23, comprising a mutation of the Gβ₅ binding site in the DEP domain.

25. A recombinant protein of claim 23, comprising a mutation of the binding site for the DEP domain in the Gβ₅ subunit.

26. A method of identifying a compound capable of modulating weight gain comprising: administering a test compound to a first mouse comprising a deletion of one allele of a gene encoding a Gβ₅ protein, and comparing the weight gain of the first mouse to the weight gain of a second mouse comprising the deletion of one allele of a gene encoding a Gβ₅ protein not administered the test compound, wherein a difference in weight gain between the first mouse and the second mouse identifies that the test compound is capable of modulating weight gain.

27. The method of claim 26, wherein the first mouse and the second mouse are Gβ₅ heterozygous mice.

28. The method of claim 26, wherein the first mouse and the second mouse are RGS7 heterozygous mice.

29. A method of identifying a compound capable of modulating the conformation of a Gβ₅ complex, comprising expressing in a host cell a first hybrid DNA sequence encoding a fusion protein comprising an RGS protein and a fluorescence acceptor or donor, and a second hybrid DNA sequence encoding a fusion protein comprising a Gβ₅ subunit and a fluorescence acceptor or donor; contacting the host cell with a test compound; exciting the fluorescence donor at a particular wavelength; detecting fluorescence emission of the acceptor; and comparing the fluorescence emission in the presence and absence of the test compound, wherein a difference in fluorescence emission identifies that the compound is capable of modulating the conformation of the Gβ₅ complex.

30. A kit for identifying a compound capable of modulating the conformation of Gβ₅ complex, comprising DNA constructs encoding the first and second hybrid DNA sequences of claim 29 and a host cell for transfection with the DNA constructs.

31. A method for predicting the onset of obesity in an individual, comprising identifying a mutation in a Gβ₅ gene and correlating the identified mutation with a prediction of the onset of obesity in an individual carrying such a mutation.

32. A method of modulating muscarinic receptor signaling in vivo comprising:
administrating to a subject an agent which modulates an interaction between a regulator of G protein signaling (RGS) and the muscarinic receptor; and,
modulating muscarinic receptor signaling in vivo.

33. The method of claim 32, wherein the agent modulates the interaction of a DEP domain of a member of an R7 family of a regulator of G protein signaling (RGS7) and muscarinic acetylcholine M3 receptor (M3R).

34. The method of claim 32, wherein the agent inhibits interaction between the DEP domain of RGS7 and muscarinic acetylcholine M3 receptor (M3R).

35. The method of claim 32, wherein the agent promotes interaction between the DEP domain of a regulator of G protein signaling (RGS7) and muscarinic acetylcholine M3 receptor (M3R).

36. A method of identifying agents which modulates interactions between a regulator of G protein signaling (RGS) and a muscarinic receptor comprising:
contacting a Gβ5-RGS7 complex and muscarinic receptor domains with libraries of molecules; and,
identifying agents which modulates interactions between a regulator of G protein signaling (RGS) and a muscarinic receptor.

37. The method of claim 36, wherein the agent modulates the interaction of a DEP domain of a member of an R7 family of a regulator of G protein signaling (RGS7) and muscarinic acetylcholine M3 receptor (M3R).

38. The method of claim 36, wherein the agent inhibits interaction between the DEP domain of RGS7 and muscarinic acetylcholine M3 receptor (M3R).

39. The method of claim 36, wherein the agent promotes interaction between the DEP domain of a regulator of G protein signaling (RGS7) and muscarinic acetylcholine M3 receptor (M3R).

40. The method of claim 36, wherein a cell comprises the Gβ5-RGS7 complex and muscarinic receptor domains.

41. The method of claim 36, wherein the interactions of the Gβ5-RGS7 complex and muscarinic receptor domains are identified by assays comprising: nucleic acid chips, peptide chips, immunoassays, blotting assays, calcium measuring assays, or gene based assays.

42. The method of claim 36, wherein the interactions of the Gβ5-RGS7 complex and muscarinic receptor domains are identified by high-throughput screening assays.

43. The method of claim 36, wherein the Gβ5-RGS7 complex and/or muscarinic receptor domains comprise at least one mutation.
